(19) United States
(12) Patent Application Publication
(10) Pub. No.: US 2014/0044648 A1
(43) Pub. Date: Feb. 13, 2014

(54) ACTIVATABLE IMAGING CONTRAST AGENTS

(71) Applicant: University of Central Florida Research Foundation, Inc., Orlando, FL (US)

(72) Inventors: J. Manuel Perez, Orlando, FL (US); Santimukul Santra, Orlando, FL (US)

(73) Assignee: University of Central Florida Research Foundation, Inc., Orlando, FL (US)

(21) Appl. No.: 13/936,933

(22) Filed: Jul. 8, 2013

Related U.S. Application Data
(60) Provisional application No. 61/668,622, filed on Jul. 6, 2012.

(51) Int. Cl. A61K 49/18

(52) U.S. Cl. CPC A61K 49/1887
USPC 424/9.323

(57) ABSTRACT

An activatable probe and methods of using the same are provided. The activatable probe includes a superparamagnetic core and a polymeric matrix coating the metal oxide core. A paramagnetic agent encapsulated within the polymeric matrix. The polymeric matrix is configured to release the paramagnetic agent when subjected to a medium having a pH less than a normal physiological pH.

### CROSS-REFERENCE TO RELATED APPLICATIONS

This application claims priority to U.S. Provisional Application No. 61/668,622, filed on Jul. 6, 2012, and which is incorporated by reference herein in its entirety.

### STATEMENT REGARDING FEDERAL FUNDING

The invention was supported in part by the National Institute of Health via NIH grant GM084331. The U.S. government has rights in this invention.

### FIELD OF THE INVENTION

The present invention relates to imaging agents, and more particularly to probes for use as contrast agents that may become activated in an environment with a pH less than a normal physiological pH.

### BACKGROUND OF THE INVENTION

Magnetic resonance imaging (MRI) has become a powerful technique in the clinical diagnosis of disease and in animal imaging.¹⁻⁴ MRI is capable of obtaining tomographic images of living subjects with high spatial resolution. It is based on the interaction of water protons with surrounding molecules within tissues in the presence of an external magnetic field.⁵⁻⁸ MR contrast agents⁹⁻¹¹ typically enhance contrast for more accurate diagnosis. Most recently, MR agents have been modified to allow for targeting imaging by conjugating targeting ligand (e.g. antibody, peptide) is conjugated to MR contrast agent.^{12,13} Among these probes, superparamagnetic nanoparticles¹⁴⁻¹⁶ and paramagnetic metal chelates⁸ are the most commonly used.¹⁷⁻²¹ Superparamagnetic nanoparticles are typically composed of an iron oxide nanoparticle (IONP) surrounded by a polymeric coating to facilitate increased stability in aqueous media.²² They work by shortening the traverse relaxation time (T₂) of surrounding water protons, resulting in a decrease of the signal (negative contrast, dark signal) using the T₂-weighted sequences for the MR scanner.²³⁻²⁸ On the other hand, paramagnetic gadolinium chelates create an increase in signal intensity on T₁-weighted images (positive contrast, bright signal) by shortening the longitudinal relaxation time (T₁) of surrounding water protons.^{10,17,29-38}

The development of an activatable MR imaging agent that reports on a biological process associated with diseases would greatly advance medical imaging of disease at a molecular level.³⁹⁻⁴¹ Activatable T₁ or T₂ agents, those that results in modulation of either the T₁ or T₂ relaxation time upon target binding, enzymatic activity or biological process associated with disease would be attractive MR imaging agents, resulting in high sensitivity and high signal to noise ratios with low background.^{20,42-48} Activatable Gd-based T₁ agents have been previously described^{8,49,50} and include those designed to be biologically activated by an enzyme such as β-Galactosidase^{51,52} and β-Glucoronidase^{42,44} as well as those activated by a release of a drug.^{53,54} Activatable T₂ IONP based agents are less common as it is often difficult to “quench” the strong superparamagnetic nature of these nanoparticles.^{26-29,55} Magnetic relaxation switches, have been developed based on IONP that cluster in the presence of a target or enzymatic activity leading to detectable changes in the T₂ relaxation times.⁵⁶⁻⁵⁹ However, the use of these T₂ activatable agents has been difficult to implement in cells or animal studies and it has been limited to their use as nanosensors in molecular diagnostic applications.^{57,60}

An activatable T₁ agent, one that can induce a faster T₁ relaxation, would result in an increase in the T₁-weighted MR signal intensity upon target recognition for better diagnosis. Such an activatable agent could be beneficial in cancer diagnosis if it were designed to become activated upon tumor targeting, resulting in a brighter signal.

### SUMMARY OF THE INVENTION

In accordance with an aspect, there is now described the design, synthesis and characterization of a novel probe that becomes activated in an environment having a less than normal physiological pH, resulting in an increase in the T₁-weighted signal (brighter contrast). In one aspect, the designed probe is composed of a superparamagnetic core, such as an iron oxide nanoparticle, that encapsulates a paramagnetic agent, such as a gadolinium and diethylenetriaminepentacetate (Gd-DTPA) chelate, within hydrophobic pockets of the nanoparticle's polymeric matrix, e.g., a polyacrylic acid (PAA) coating (IO-PAA-Gd-DTPA). While not wishing to be bound by theory, it is believed that the strong magnetic field of the superparamagnetic iron oxide core will affect the relaxation process of the much weaker paramagnetic Gd-DTPA, resulting in quenching of its T₁ signal (FIG. 1). The present inventors observed, for example, that the T₁ relaxation rate (1/T₁) of the Gd(III)-DTPA complex was quenched (OFF/Dark) when the Gd-DTPA complex was encapsulated within the PAA coating of the iron oxide nanoparticle (IO-PAA). Upon release of the quenched Gd-DTPA, an increase in the T₁ relaxation rate was observed with marginal increase in the T₂ relaxation rate (1/T₂). This quenching effect was not observed when the Gd chelate was attached to the surface of the IONP or when a non-magnetic nanoparticles, such as cerium oxide nanoparticles, were used to encapsulate the Gd-DTPA. Corresponding R₁ and R₂ values for the IO-PAA-Gd-DTPA nanocomposite at different pH revealed a pH-dependent increase in the R₁ of the nanocomposite suspension as the pH decreases, indicating T₁ activation at acidic pH. The observed pH dependent increase in R₁ was only observed when Gd-DTPA was encapsulated within the polymeric coating of the nanoparticle, but not when Gd-DTPA was directly attached on the surface of the nanoparticle's polymeric coating.

In addition, the present inventors have found that the superparamagnetic iron oxide nanocrystal acted as a magnetic quencher for the Gd-DTPA T₁ only when the Gd-DTPA is encapsulated within the nanoparticle's polymeric coating in close proximity to the superparamagnetic core. Also, it was confirmed that the T₂ activation of the probes was not quenched upon encapsulation of Gd-DTPA complex. Furthermore, when the IO-PAA-Gd-DTPA nanocomposite was conjugated with a targeting agent, such as folic acid, its selective internalization and lysosomal localization within folate receptor positive cells allow for selective activation due to the lysosome's acidic pH. Still further, when the folate receptor targeting nanocomposite was used to co-encapsulate a cytotoxic drug (e.g., Taxol), dual delivery of the drug and T₁ imaging activation was achieved. Taken together, the newly developed activatable probes (IO-PAA-Gd-DTPA) combine features of several important modalities, such as: (i) activatable T1-weighted MRI contrast, (ii) T₂-weighted MRI contrast, (iii) receptor-targeted internalization, (iv) biodegradable and biocompatible and/or (v) tumor delivery of anticancer drug(s). These features render the described probes as particularly suitable MR-activatable agents for cancer.

### BRIEF DESCRIPTION OF THE DRAWINGS

FIG. 1 is a schematic representation of the acidic pH-mediated activation of the activatable composite magnetic nanoprobe IO-PAA-Gd-DTPA and corresponding T₁-MR activation.

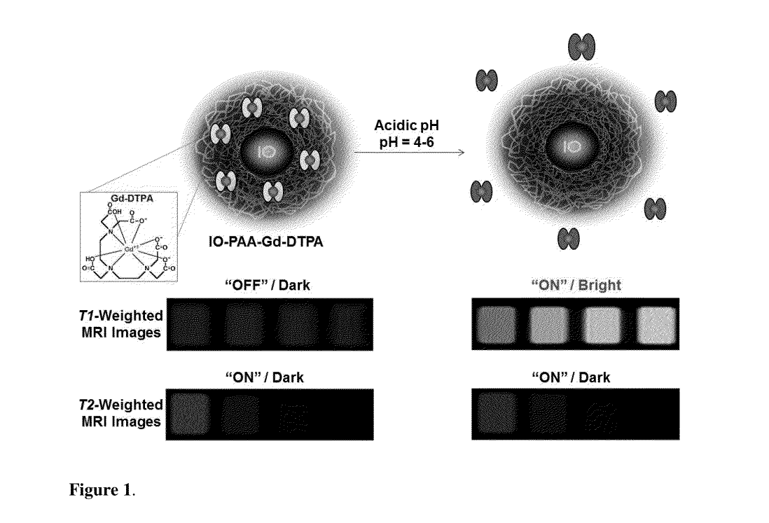

FIGS. 2A-2D show the measurement of hydrodynamic diameter by dynamic light scattering (DLS) and the overall size by scanning transmittance electron microscopy (STEM, scale bar 200 nm, Inset) of A) the control probe (IO-PAA) and B) the activatable probe (IO-PAA-Gd-DTPA). C) FT-IR spectra showing successful PAA coating, whereas D) the overall surface charge (zeta potential) of different functional magnetic nanoprobes (carboxylated nanoprobe: −41 mV, alkynated nanoprobe: −16 mV and folate nanoprobe: −29 mV) were measured using zeta seizer, indicating successful surface functionalization of our magnetic nanoprobes.

FIG. 3 is a schematic representation of the acid-mediated magnetic relaxations of the composite nanoceria NC-PAA-Gd-DTPA nanoprobe and the change in magnetic relaxations was shown by the corresponding T₁-weighted MRI (B=4.7 T) images. DLS and ICP-MS of the nanoprobe aqueous suspension indicated the presence of 88±1 nm nanoparticles with a Gd concentration of 0.315 mg/mL.

FIGS. 4A-4F show an assessment of magnetic relaxations of activatable magnetic nanoprobe IO-PAA-Gd-DTPA using bench-top magnetic relaxometer (Bruker's Minispec, B=0.47 T). Inverse spin-lattice (1/T₁) and spin-spin (1/T₂) magnetic relaxation times were measured before and after 24 h of incubation in different PBS solutions (pH=4.0-7.4, 37° C.) and at different nanoprobe concentrations. (A) Initial 1/T₁ measurements right after the addition of PBS solutions, (B) 1/T₁ measurements after 24 h of incubation, (C) the differential 1/T₁ values prior to and after incubation. (D) Initial 1/T₂ measurements right after the addition of PBS solutions, (E) 1/T₂ measurements after 24 h of incubation, (F) the differential 1/T₂ values prior to and after incubation.

FIGS. 5A-5F show an assessment of magnetic relaxations of control magnetic nanoprobe IO-PAA using bench-top magnetic relaxometer (Bruker's Minispec, B=0.47 T). Inverse spin-lattice (1/T₁) and spin-spin (1/T₂) magnetic relaxation times of control IO-PAA nanoprobe were measured before and after 24 h of incubation in different PBS solutions (pH=4.0-7.4, 37° C.) and at different Fe concentrations. (A) Initial 1/T₁ measurements of IO-PAA nanoprobes right after the addition of PBS solutions, (B) 1/T₁ measurements after 24 h of incubation, (C) The differential 1/T₁ values prior to and after incubation, (D) Initial 1/T₂ measurements of IO-PAA nanoprobes right after the addition of PBS solutions, (E) 1/T₂ measurements after 24 h of incubation, (F) The differential 1/T₂ values prior to and after incubation.

FIGS. 6A-6F show an assessment of magnetic relaxations of composite nanoceria NC-PAA-Gd-DTPA using bench-top magnetic relaxometer (Bruker's Minispec, B=0.47 T). Inverse spin-lattice (1/T₁) and spin-spin (1/T₂) magnetic relaxation times were measured before and after 24 h of incubation in different PBS solutions (pH=4.0 and 7.4, 37° C.) and at different nanoprobe concentrations. (A) Initial 1/T₁ measurements right after the addition of PBS solutions, (B) 1/T₁ measurements after 24 h of incubation and (C) the differential 1/T₁ values prior to and after incubation. (D) Initial 1/T₂ measurements right after the addition of PBS solutions, (E) 1/T₂ measurements after 24 h of incubation and (F) the differential 1/T₂ values prior to and after incubation.

FIG. 7 is a schematic representation of the Gd-DTPA surface conjugating IO-PAA magnetic nanoprobe, IO-PAA-Gd-DTPA-Surface and the corresponding changes in acid-mediated magnetic relaxations of the Gd-DTPA surface conjugating IO-PAA magnetic nanoprobe, as shown by the T₁- and T₂-weighted MRI (B=4.7 T) images.

FIGS. 8A-8F show an assessment of magnetic relaxations of nanoceria NC-PAA using bench-top magnetic relaxometer (Bruker's Minispec, B=0.47 T). Inverse spin-lattice (1/T1) and spin-spin (1/T₂) magnetic relaxation times of NC-PAA nanoprobe were measured before and after 24 h of incubation in different PBS solutions (pH=4.0 and 7.4, 37° C.) and at different nanoceria concentrations. (A) Initial 1/T₁ measurements of NC-PAA nanoprobes right after the addition of PBS solutions, (B) 1/T₁ measurements after 24 h of incubation, (C) The differential 1/T₁ values prior to and after incubation, (D) Initial 1/T₂ measurements of NC-PAA nanoprobes right after the addition of PBS solutions, (E) 1/T₂ measurements after 24 h of incubation, (F) The differential 1/T₂ values prior to and after incubation.

FIGS. 9A-9F show an assessment of magnetic relaxations of Gd-DTPA surface conjugating IO-PAA magnetic nanoprobes, using bench-top magnetic relaxometer (Bruker's Minispec, B=0.47 T). Inverse spin-lattice (1/T₁) and spin-spin (1/T₂) magnetic relaxation times were measured before and after 24 h of incubation in different PBS solutions (pH=4.0-7.4, 37° C.) and at different nanoprobe concentrations. (A) Initial 1/T₁ measurements right after the addition of PBS solutions, (B) 1/T₁ measurements after 24 h of incubation and (C) the differential 1/T₁ values prior to and after incubation. (D) Initial 1/T₂ measurements right after the addition of PBS solutions, (E) 1/T₂ measurements after 24 h of incubation and (F) the differential 1/T₂ values prior to and after incubation.

FIG. 10A-10D show Magnetic Resonance Imaging (MRI) studies measuring the magnetic activations (T₁- and T₂-maps) of activatable magnetic IO-PAA-Gd-DTPA nanoprobes in PBS at pH 5.0. (A) T₁-weighted MRI images of increasing Gd concentrations (0.06 μM-2.4 μM) of IO-PAA-Gd-DTPA nanoprobes prior to (1) and after 24 h of incubation (2) at 37° C., (B) T₂-weighted MRI images of increasing Fe concentrations (0.3 mM-11.5 mM) of IO-PAA-Gd-DTPA nanoprobes prior to (1) and after 24 h of incubation (2) at 37° C., (C) Corresponding 1/T₁ relaxation rates prior to () and after (▴) 24 h of incubation, (D) Corresponding 1/T₂ relaxation rate prior to () and after (▴) 24 h of incubation.

FIGS. 11A-11D show Magnetic Resonance Imaging (MRI) studies measuring the magnetic activations (using T₁- and T₂-maps) of control magnetic IO-PAA nanoprobes in PBS at pH 5.0. (A) Images from T₁-map MRI experiments of Fe increasing concentrations (0.3 mM-11.5 mM) of IO-PAA nanoprobes prior to (1) and after 24 h of incubation (2) at 37° C., (B) Images from T₂-map MRI experiments of increasing Fe concentrations of IO-PAA nanoprobes prior to (1) and after 24 h of incubation (2) at 37° C., (C) Magnetic relaxations (1/T₁) obtained by translating the corresponding MR signals into the inverse spin-lattice magnetic relaxations (1/T₁) prior to () and after (▪) 24 h of incubation, (D) No magnetic activations (1/T₂) observed by translating the corresponding MR signals into the inverse spin-spin magnetic relaxations (1/T₂) prior to () and after (▪) 24 h of incubation. This is due to the absence of any T₁ and T₂ activation of the control IO-PAA nanoprobes.

FIG. 12A-12D show intracellular magnetic activations of our folate-decorated activatable IO-PAA-Gd-DTPA-Fol nanoprobe (, 100 μL, 28 mM) and the control IO-PAA-Fol nanoprobe (▪, 100 μL, 28 mM) using FR-expressing HeLa cells (A and B) and FR-negative H9c2 cells (C and D). Significant activation in inverse spin-lattice magnetic relaxations (1/T₁) was observed from HeLa cells incubated with the activatable IO-PAA-Gd-DTPA-Fol nanoprobes (, FIG. 12A). As expected, no significant changes in 1/T₂ were observed from HeLa cells due to absence of any T2 activations (FIG. 12B). Neither 1/T₁ (FIG. 12C) nor 1/T2 (FIG. 12D) activations were observed from H9c2 cells due to lack of any receptor-mediated internalizations.

FIGS. 13A-13B show the rate of release of taxol and Gd-DTPA at 37° C. A) HPLC experiment (λ_{abs}=227 nm) indicated the time-dependent release of taxol from the activatable IO-PAA-Gd-DTPA nanoprobes (50 μL, 28 mM) when incubated at pH=5.0 (▴) solution. No significant release of taxol was observed (▪) when incubated in PBS at pH 7.4. B) The observed increase rate of taxol release was accompanied by a gradual increase in the inverse spin-lattice magnetic relaxation (1/T₁) recorded using magnetic relaxometer (▾, B=0.47 T, pH=5.0). As expected, nominal increase in the inverse spin-lattice magnetic relaxation (, 1/T₁) was observed when incubated in PBS at pH=7.4.

FIGS. 14A-14B show the low-pH mediated magnetic activation corroborated the rate of encapsulated drug release at 37° C. A) HPLC experiment (λ_{abs}=227 nm) indicated the time-dependent release of taxol from the activatable IO-PAA-Gd-DTPA nanoprobes (50 μL, 28 mM) when incubated in acidic PBS (▪, pH=5.0) solution. No significant release of taxol was observed () when incubated in serum, confirming nanoprobe's stability in serum. B) The observed increase rate of taxol release was accompanied by a gradual increase in the inverse spin-lattice magnetic relaxation (1/T₁) recorded using magnetic relaxometer (▾, B=0.47 T, pH=5.0). As expected, nominal increase in the inverse spin-lattice magnetic relaxation (, 1/T₁) was observed when incubated in serum.

FIGS. 15A-15B show time-dependent in vitro MTT assays for the determination of cytotoxicity of the functional magnetic nanoprobes (1-5, 35 μL, 28 mM in PBS pH=7.4). HeLa cells (A) and H9c2 cells (B) treated with the functional magnetic nanoprobes. Folate-conjugated (1), Gd-DTPA encapsulating (2), Gd-DTPA and taxol-encapsulating (3) magnetic nanoprobes showed biocompatibility with nominal toxicity in both the cell lines. The Gd-DTPA encapsulating folate-conjugated magnetic nanoprobes (4) showed more than 15% reduction in cell viability, whereas Gd-DTPA and taxol encapsulating folate-conjugated magnetic nanoprobes (5) showed more than 90% reduction in cell viability when treated with HeLa cells (A) and not with H9c2 cells (B), confirming the folate-receptor mediated internalizations and ability for targeted therapy. Average values of four measurements are depicted ±standard errors.

FIG. 16 shows the structure of an IO-PAA-Doxorubicin-S-S-Gd DTPA nanoprobe in accordance with an aspect of the present invention.

FIGS. 17A-F show the assessment of magnetic relaxations of activatable magnetic nanoprobe IO-PAA-Doxorubicin-S-S-Gd-DTPA using bench-top magnetic relaxometer (Bruker's Minispec, B=0.47 T). Inverse spin-lattice (1/T₁) and spin-spin (1/T₂) magnetic relaxation times were measured before and after 24 h of incubation in different PBS solutions (pH=4.0-7.4, 37° C.) and at different nanoprobe concentrations. (A) Initial 1/T₁ measurements right after the addition of PBS solutions, (B) 1/T₁ measurements after 24 h of incubation, (C) the differential 1/T₁ values prior to and after incubation. (D) Initial 1/T₂ measurements right after the addition of PBS solutions, (E) 1/T₂ measurements after 24 h of incubation, (F) the differential 1/T₂ values prior to and after incubation.

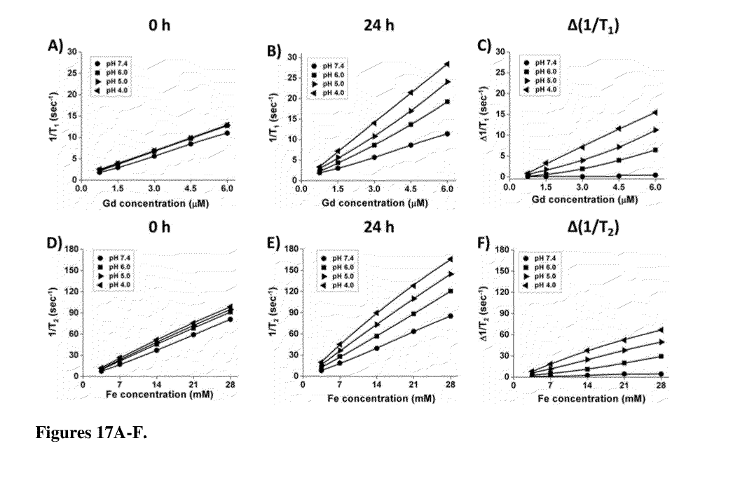

### DETAILED DESCRIPTION OF THE INVENTION

According to an aspect of the present invention, there is provided an activatable probe comprising a superparamagnetic core and a polymeric matrix coating the metal oxide core. A paramagnetic agent is encapsulated within the polymeric matrix. The polymeric matrix is configured to release the paramagnetic agent when subjected to a medium having a pH less than a normal physiological pH.

In another aspect, there is provided an activatable probe that includes the following characteristics: (i) an activatable T₁-weighted MRI contrast; (ii) a T₂-weighted MRI contrast; (iii) receptor-targeted internalization; (iv) biodegradable and biocompatible; and/or (v) tumor delivery of anticancer drug.

In another aspect, there is provided is an activatable probe comprising the following components: (a) a metal particle; (b) Gd-DTPA; and (c) a polymeric matrix; wherein the Gd-DTPA is associated with the polymeric matrix. In another embodiment, there is provided an iron oxide particle associated with Gd-DTPA via a polymeric, e.g., PAA, matrix.

In another aspect, there is provided an activatable probe comprising the following components: (a) a metal particle; (b) Gd-DTPA; (c) an anti-tumor agent and/or anti-cancer agent; and (d) a polymeric matrix, wherein the Gd-DTPA is associated with the polymeric matrix. The probe is activatable when subjected to a less than normal physiological pH. When subjected to such environment, the GD-DTPA and/or the anti-tumor and/or anti-cancer agent is released.

In yet another aspect, there is provided an anti-tumor agent and/or anti-cancer agent conjugated with the GD-DTPA component. In a particular embodiment, there is provided a Doxorubicin-S-S-Gd-DTPA activatable nanoprobe. It will be appreciated by those skilled in the art that the Doxorubicin component can be substituted with another anti-tumor agent or anti-cancer agent.

In yet another aspect, there is provided a method of enhancing imaging sensitivity of cancer tissue in a subject. The method includes administering to the subject an effective amount of an activatable probe as disclosed herein, and subjecting the subject to an imaging technique. Typically, the imaging technique pertains to MRI.

In yet another aspect, there is provided a method of imaging release of a biologically active agent in a subject. The method involves administering to the subject a therapeutically effective amount of an activatable probe disclosed herein that includes: (a) a metal particle; (b) Gd-DTPA; (c) an anti-tumor agent and/or anti-cancer agent; and (d) a polymeric matrix, wherein the Gd-DTPA is associated with the polymeric matrix. The method also involves subjecting the subject to an imaging technique. The imaging technique typically involves MRI.

As used herein, the term “about” refers to values that are ±10% of the stated value.

As used herein, the terms “administering,” “administration,” or the like includes any route of introducing or delivering to a subject a composition (e.g., pharmaceutical composition or wound dressing) to perform its intended function. The administering or administration can be carried out by any suitable route, including topically, orally, intranasally, parenterally (intravenously, intramuscularly, intraperitoneally, or subcutaneously), rectally, or topically. Administering or administration includes self-administration and the administration by another.

As used herein, the term “anti-cancer agent” refers to any biologically effective agent that has an anti-cancer effect on a cell in a subjecting, including but not limited a cytotoxic effect, an apoptotic effect, an anti-mitotic effect, an anti-angiogenesis effect, or an anti-metastatic effect.

As used herein, the term “biologically effective agent” refers to any material used to treat or prevent any disease, disorder or abnormal condition in a subject.

As used herein, the term “cancer” refers to all types of cancers or neoplasm or malignant tumors found in a subject.

As used herein, the terms “effective amount,” “amount effective,” “therapeutically effective amount,” or the like, refer to an amount effective at dosages and for periods of time necessary to achieve the desired result.

As used herein, the term “paramagnetic material” is meant to include any material which possesses a magnet moment that can be aligned by an external magnetic field. In the probes described herein, the T₁ relaxation rate of the paramagnetic agent as described herein is quenched by the superparamagnetic core to at least some extent.

As used herein, the term “subject” refers to any human or nonhuman mammal.

As used herein, the term “superparamagnetic” refers to a class of substances that have a similar magnetism as ferromagnetic materials in an external magnetic field, but do not have a remnant magnetization after removal of the external magnetic field. Typically, superparamagnetic agents work by shortening the traverse relaxation time (T₂) of surrounding water protons, resulting in a signal decrease using the T₂-weighted sequences for the magnetic resonance scanner. In the nanoprobes described herein, the superparamagnetic materials have an ability to at least quench (reduce) the T₁ relaxation rate of the paramagnetic agent as described herein to at least some extent.

The superparamagnetic core may comprise any suitable material having an ability to at least quench (reduce) the T₁-weighted signal of the paramagnetic agent as described herein to at least some degree. In one aspect, the superparamagnetic material comprises a metal. The metal may comprise a compound comprising at least one of the group consisting of Au, Ag, Pd, Pt, Cu, Ni, Co, Fe, Mn, Ru, Rh, Os, and Ir, for example. In one embodiment, the superparamagnetic core comprises a superparamagnetic iron platinum particle (SIPP). In another embodiment, the superparamagnetic core comprises a metal oxide, including but not limited to a member from the group consisting of zinc oxide, titanium dioxide, iron oxide, silver oxide, copper oxide, aluminum oxide, and silicon dioxide particles. In a particular embodiment, the superparamagnetic core comprises iron oxide. The core of the probe may be in any suitable form, such as a magnetic bead, nanoparticle, microparticle, and the like.

In certain embodiments, the superparamagnetic core comprises a nanoparticle having a longest dimension of less than about 1000 nm, and in certain embodiments less than 100 nm. In addition, in certain embodiments, the probes described herein comprise nanoprobes, even when the remaining components described herein are included with the nanoparticle (e.g., polymeric matrix, paramagnetic agent, targeting agent, and/or biologically active agent). In other embodiments, the superparamagnetic core is a micron-sized particle and the corresponding probes are micron-sized.

The polymeric matrix may be any polymeric material that degrades and/or swells at a pH less than normal physiological conditions (typically about 7.4). In this way, in the probes described herein, the polymeric matrix can release the agents contained therein, such as a paragmagnetic agent, targeting agent and/or biologically active agent at pH's less than a normal physiological pH. In certain embodiments, the polymeric matrix comprises a polymeric material that degrades and/or swells at a pH within the range of about 4.0 to about 7.4. In a particular embodiment, the polymeric matrix comprises a polymeric material that degrades and/or swells at a pH within the range of about 5.0 to about 6.0. It is appreciated that at normal pH's, the polymeric matrix effectively encapsulates the cargo so as to substantially maintain the cargo therein. By “encapsulate,” it is meant that at least a portion of the cargo (paramagnetic agent, targeting agent, and/or biologically active agent) is encapsulated within the polymeric matrix, e.g., not on a surface of the matrix. Exemplary polymeric materials include but are not limited to polyacrylic acid, dextran, and chitosan. In a particular embodiment, the polymeric matrix comprises polyacrylic acid (PAA).

The paramagnetic agent may comprise any material that whose T₁ signal may be quenched by the superparamagnetic core in a probe as described herein to at least some degree. The paramagnetic agent may include a member selected of the transition metals and lanthanides of groups 1b, 2b, 3a, 3b, 4a, 4b, 5b, 6b, 7b, and 8. In certain embodiments, the paramagnetic agent comprises a member from the group consisting of gadolinium (Gd), dysprosium (Dy), chromium (Cr), and manganese (Mn). In a particular embodiment, the paramagnetic agent comprises Gd.

In one aspect, the paramagnetic agent also comprises a chelating moiety, capable of forming chelate-complexes with the paramagnetic agent. Exemplary chelating moieties include but are not limited to diethylenetriamine pentaacetic acid (DTPA), ethylene diamine tetraacetic acid (EDTA), triethylene tetraamine hexaacetic acid (TTHA), tetraethylene pentaamine heptaacetic acid, and polyazamacrocyctic compounds, such as 1,4,7,10-tetra-azacyclododecane-1,4,7,10-tetraacetic acid (DOTA)]. In certain embodiments, as described herein, the paramagnetic agent comprises a GD-DTPA complex.

The polymeric matrix is effective to release its cargo when subjected to a medium having a pH less than a normal physiological pH and to encapsulate the cargo within its polymeric matrix at normal physiological pH. In this way, the polymeric matrix can be tuned to release an effective amount of its cargo as desired for the particular application. The present inventors have found that the probes described herein are particularly useful for imaging cancer cells that have a pH environment less than a normal physiological pH. This is particularly advantageous as it is becoming of increasing interest that tumor cell survival relies on adaptation to acidic conditions in the tumor microenvironment. In fact, it has been found that the physiological relevant pH range in certain tumor cells, including but not limited to breast, lung, cervical, and pancreatic cancer cells, is about pH 5 to pH 6. As such, the probes described herein are particularly suitable for imaging such tumor cells or monitoring delivery of biological agents thereto. The cancer cells suitable for targeting and/or imaging are without limitation so long as they produce a microenvironment that has a pH less than a normal physiological pH.

To render the probes selective for imaging particular cells or tissue, in one aspect, the probes may further include a targeting agent having an affinity for a predetermined molecular target, such as a cell receptor. In certain embodiments, the targeting agent is also encapsulated within the polymeric matrix along with the paramagnetic agent. In one embodiment, for example, the targeting agent comprises a folate targeting compound that targets cancer cells that overexpress folate receptors. The folate targeting compound may comprise folate, folic acid, or derivatives thereof. Examples of folate derivatives include, but are not limited to, dihydrofolate, tetrahydrofolate, 5,-methyl-tetrahydrofolate and 5,10-methylene tetrahydrofolate. Humans and other mammals express a number of proteins which bind to folate and transport it into cells. For example, in humans, alpha- and beta-folate receptors have been identified, each of which can occur in several isoforms (e.g. as a result of differential glycosylation). These proteins are referred to as “folate receptors.” Thus, a folate receptor is considered to be any protein expressed on the surface of a cell, such as a cancer cell, which binds the folate targeting compound in preference to other moieties or compounds.

Additionally, in other embodiments, the targeting agent may be one or more of an aptamer, a peptide, an oligonucleotide, an antigen, an antibody, or combinations thereof having an affinity for a predetermined molecular target. In one embodiment, the targeting agent comprises an aptamer having an affinity for a cancer cell. The aptamer may include any polynucleotide- or peptide-based molecule, for example. A polynucleotidal aptamer is a DNA or RNA molecule, usually comprising several strands of nucleic acids that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

In addition to the targeting agent or in lieu thereof, the probes described herein may include a biologically active agent encapsulated within the polymeric matrix. Since some cancer cells are particularly useful targets due to their reduced pH microenvironments, the biologically active agent may be an anti-cancer agent in certain embodiments. The composition of the anti-cancer agent is without limitation as the anti-cancer agent is typically only encapsulated within the polymeric matrix, and not bonded thereto. Exemplary anti-cancer agents are disclosed in U.S. Published Application No. 20130045949, the entirety of which is incorporated by reference herein. In a particular embodiment, the anti-cancer agent is selected from the group consisting of taxol and doxorubicin.

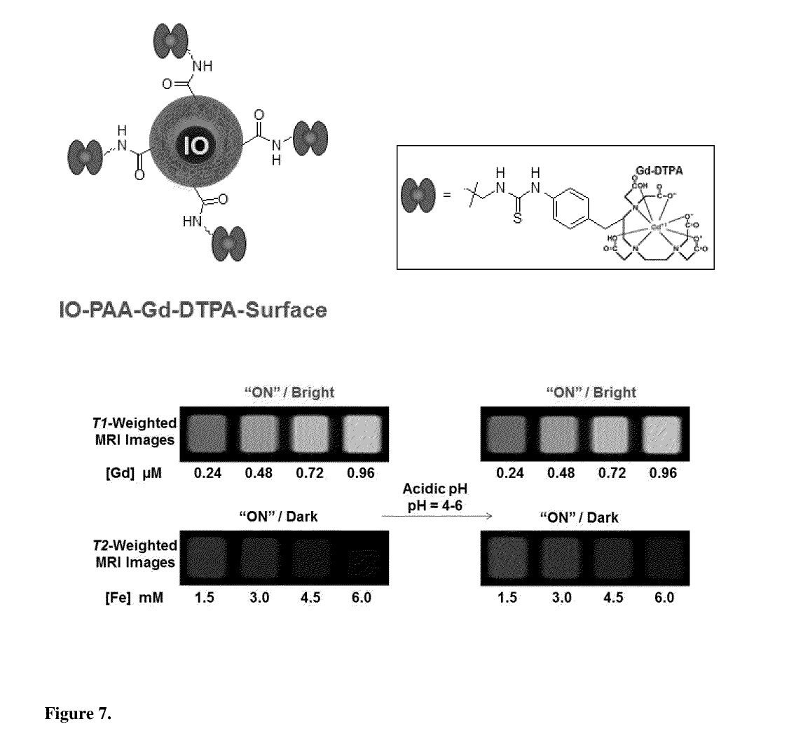

In certain embodiments, the targeting agent and/or the biologically active agent are bonded (covalently or ionically, and typically covalently) to the paramagnetic complex. For example, as shown in the examples, there is provided an 10 (iron oxide)-doxorubicin-S-S-Gd-DTPA probe wherein the doxorubicin molecule is bonded to the Gd-DTPA complex via a disulfide bond. The disulfide bond is expected to be broken down upon release of the doxorubicin-Gd DTPA complex from the polymeric matrix under normal physiological conditions or conditions having a pH lower than the normal physiological conditions. This approach guarantees that upon the release of the drug (Doxorubicin), activation of the MR signal will occur indicating assessment of drug release by MRI. In an embodiment, the cleavable “Doxo-S-S-Gd-DTPA” conjugate was synthesized using a facile nucleophilic substitution reaction before encapsulating with IO-PAA as described earlier in the case of IO-PAA-Gd-DTPA. In a typical reaction, the aqueous solution of doxorubicin hydrochloride salt (1.75 mmol) was added to PBS buffer solution (pH=8.4) to obtain doxorubicin with free amine group. The resulting solution was centrifuged and the solid pallet was soluble in DMSO. Then, p-NH₂-Bn-Gd-DTPA complex (1.75 mmol, in PBS, pH 7.4) and dithiobis(succinimidyl propionate) (DSP) solution (1.75 mmol, in DMSO) were added drop-wise. A catalytic amount of triethylamine (0.5 μL in DMSO) was added to the reaction mixture. The reaction mixture was incubated at room temperature for 30 minutes, before overnight incubation at 4° C. (FIG. 7). The final product “Doxo-S-S-Gd-DTPA” was purified following chromatographic methods and kept at 4° C. as stock solution.

The probes herein can be utilized for enhancing imaging sensitivity of tissue in a subject comprising by administering to the subject an effective amount of an activatable probe for a time sufficient to release the paramagnetic agent from the polymeric matrix, and subjecting the subject to an imaging technique, typically an MRI technique. For example, in an embodiment, the activatable probe may be administered intravenously into the subject either prior to or during an MRI examination, such as by hypodermic injection or by catheter. In one embodiment, the administration site is at or adjacent to the site where the examination is to be made. In another embodiment, the probe is transferred to the site of examination, such as via the bloodstream.

One skilled in the art would readily appreciate that the administration, duration, and dosing (e.g., concentration) of the components of the probes/compositions described herein may be determined or adjusted based on the age, body weight, general condition, sex, diet, and/or the intended use thereof. Effective amounts of the probes can be provided in a single administration or multiple administrations. When administering the probes described herein, the imaging amount may range from 3 to 50 milliliters in a suitable concentration, for example, depending upon the purpose of the administration. Once administered, the imaging may be performed by suitable methods and devices as known in the art. Exemplary MR imaging methods and devices are disclosed in D. M. Kean and M. A. Smith, Magnetic Resonance Imaging: Principles and Applications (William and Wilkins, Baltimore 1986); U.S. Pat. Nos. 6,151,377, 6,144,202, 6,128,522, 6,127,825, 6,121,775, 6,119,032, 6,115,446, 6,111,410 and U.S. Published Patent Application No. 20110200534, the entirety of each of which is hereby incorporated herein by reference.

In addition, in another aspect, there are provided methods of imaging a release of a biologically active agent in a subject comprising administering to the subject an effective amount of an activatable probe as described herein, and subjecting the subject to an imaging technique, typically an MRI technique.

The following examples are provided as an aid in examining particular aspects of the invention, and represent only certain embodiments and explanations of embodiments. The examples are in no way meant to be limiting of the invention scope. The materials and methods provided below are those which were used in performing the examples that follow.

### EXAMPLES

### 1.0 Results

### 1.1 Synthesis and Characterization of Gd-DTPA Composite Iron Oxide Nanoparticles.

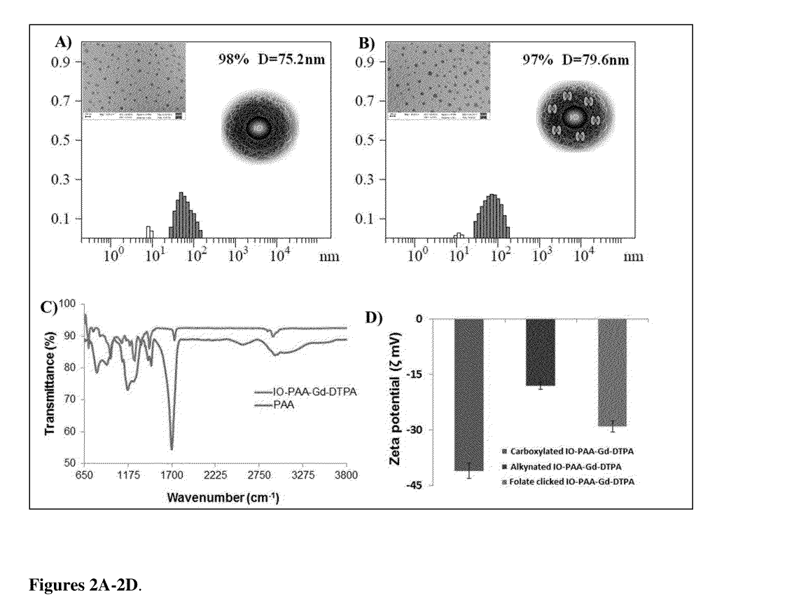

A IO-PAA-Gd-DTPA probe was synthesized by direct addition of Gd-DTPA during the course of the IO-PAA synthesis using a modified version of a published protocol.²² In brief, an aqueous solution of PAA (0.45 mmol) and Gd-DTPA (0.04 mmol) was added and mixed thoroughly before addition of a mixture of iron salts (2.26 mmol of FeCl₃.6H₂O and 1.61 mmol of FeCl₂.4H₂O in dilute HCl solution) in aqueous ammonium hydroxide solutions (0.05 M). The resulting dark-brown colored suspension of composite IO-PAA-Gd-DTPA nanoprobe was stirred for 1 h at room temperature and then centrifuged at 4000 rpm for 30 minutes to get rid of free polyacrylic acid, not encapsulated Gd-DTPA complex and other unreacted reagents. Finally, the composite nanoprobe suspension was purified using a magnetic column (Miltenyi Biotech) and washed with phosphate buffer saline (pH=7.4) solution. This “in situ” encapsulation approach proved to be effective for the encapsulation of Gd-DTPA as no change in the size and relaxivity of the nanoprobes were found over the long period of time (Table 1). The encapsulation of Gd-DTPA within the nanoprobe was confirmed by measuring the amount of Gd using ICP-MS (0.289 mg Gd/mL). Magnetic relaxation measurements at 0.47 T of the composite nanoprobes resulted in a Gd-concentration based relaxivity of R₁=50.2±1.8 mM⁻¹Sec⁻¹ and R₂=87.3±2.4 mM-1 Sec-1; and R₁=43.3±2.1 mM⁻¹Sec⁻¹ and R₂=230±3 mM⁻¹Sec⁻¹ based on Fe concentration. Dynamic light scattering studies indicated the presence of a stable and monodisperse suspension of nanoparticles with a hydrodynamic diameter of D 79±2 nm. The diameters of these magnetic nanoprobes were further confirmed by scanning transmittance electron microscopic (STEM) experiments, which show an average diameter of 80 nm (FIG. 2). The synthesized IO-PAA-Gd-DTPA nanocomposite was found to be stable in PBS (pH=7.4) and in serum, as no binding, clustering or precipitations of the nanoparticles were observed over the long period of time. Similarly, the stability of the composite nanoparticles was further confirmed by observing no significant changes in magnetic relaxations, as shown in Table 1 below. Taken together, these results indicate the effective encapsulation of Gd-DTPA into the IO-PAA polymeric matrix.

TABLE 1
Magnetic relaxations and size of the magnetic nanoprobes were
measured using 0.47 T magnet in physiological conditions. R1
and R2 values are calculated based on Fe concentrations.

| | IO-PAA | IO-PAA-Gd-DTPA | Time |
|---|---|---|---|
| R1/R2 | 32 ± 1/251 ± 2 | 43 ± 1/230 ± 2 | 15 Days |
| D (PDI) | 75 ± 1 (0.81) | 79 ± 2 (0.92) | |
| R1/R2 | 33 ± 1/253 ± 2 | 44 ± 1/232 ± 3 | 1 Month |
| D (PDI) | 75 ± 2 (0.85) | 80 ± 2 (0.90) | |
| R1/R2 | 34 ± 2/253 ± 2 | 43 ± 2/232 ± 2 | 3 Months |
| D (PDI) | 77 ± 1 (0.93) | 81 ± 1 (0.86) | |
| R1/R2 | 34 ± 1/255 ± 1 | 42 ± 1/231 ± 3 | 6 Months |
| D (PDI) | 76 ± 2 (0.88) | 81 ± 2 (0.92) | |
| R1/R2 | 35 ± 2/254 ± 3 | 42 ± 2/235 ± 2 | 1 Year |
| D (PDI) | 79 ± 2 (0.94) | 83 ± 1 (0.89) | |

Table 1: Both the activatable magnetic nanoprobe (IO-PAA-Gd-DTPA) and the control probe (IO-PAA) were found to be highly stable in 1X PBS (pH = 7.4) and in serum. Experimental results showed that the synthesized nanoprobes were highly stable in both aqueous buffered solution (PBS, pH = 7.4) and in serum for more than a year, without significant precipitation (no significant change in size) or changes in magnetic relaxations.

1.2 pH-Dependent Activation of the Gd-DTPA Composite Magnetic Nanoprobes.

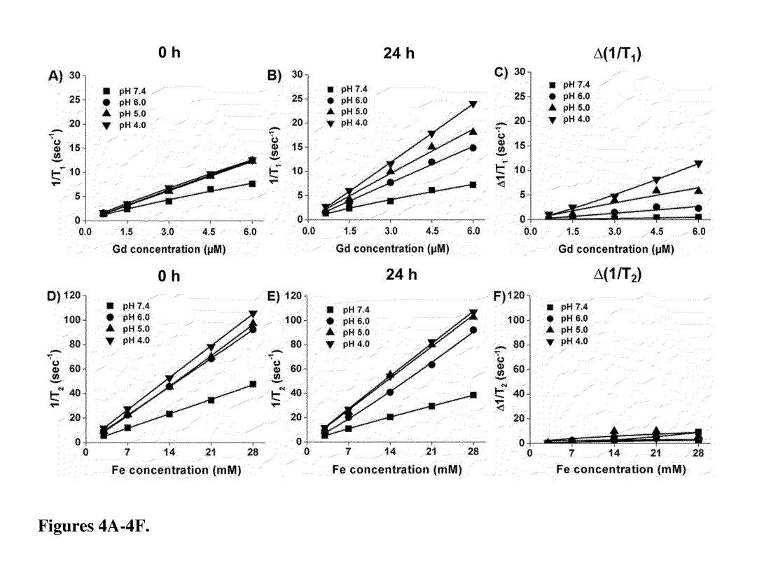

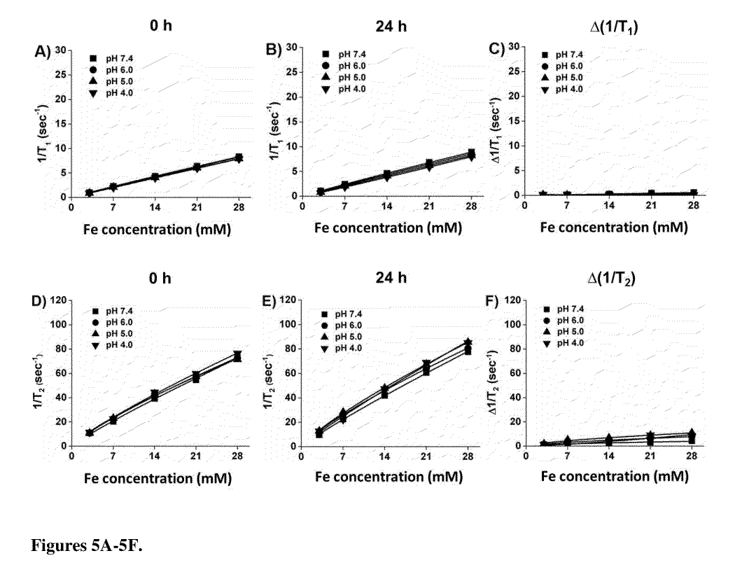

The magnetic relaxation activation of the IO-PAA-Gd-DTPA nanoprobes in buffered solution within a pH range of 4.0 to 7.4 was evaluated. In these experiments, the T₁ and T₂ of increasing concentrations of IO-PAA-Gd-DTPA nanoprobes was measured at physiological (pH=7.4) and acidic (pH=4.0-6.0) buffered solutions. T₁ and T₂ readings were taken upon addition of the magnetic nanoprobes, immediately (0 h) and after a 24 h of incubation of the magnetic nanoprobes in the corresponding buffered solutions at 37° C. First, it was observed that the T₁ relaxation rate (1/T₁) of the IO-PAA-Gd-DTPA nanoprobe (0 h, FIG. 4A) was similar to that of the control IO-PAA nanoprobe (0 h, FIG. 5A) at all pH values (pH 4.0-7.4). This observation seems to indicate that in the IO-PAA-Gd-DTPA nanoprobe the 1/T₁ of Gd-DTPA was quenched upon encapsulation in the polymeric coating of IO-PAA. In contrast, a greater increase in 1/T₁ of the IO-PAA-Gd-DTPA nanoprobe was observed when incubated in acidic [pH=4.0 (▾), 5.0 (▴) and 6.0 ()] buffered solution after 24 h (FIG. 4B). However, no changes in 1/T₁ were observed either for IO-PAA-Gd-DTPA when incubated at physiological pH over the same 24 h time period (pH=7.4, ▪, FIG. 4B) or for equivalent concentrations of control IO-PAA across the same pH values (pH=4.0 to 7.4) after 24 h of incubation (FIG. 5B). These results suggest that the composite IO-PAA-Gd-DTPA nanoprobe gets activated, resulting in high Δ1/T₁ numbers (FIG. 4C) within 24 h of incubation in the acidic buffered solutions in contrast to values obtained with the control IO-PAA probe (FIG. 5C). In another set of experiments, minimal changes in T₂ relaxation rate (Δ1/T₂) were observed for both the composite IO-PAA-Gd-DTPA nanoprobe (FIG. 4D-F) and control IO-PAA nanoprobe (FIG. 5F) when incubated for 24 h in buffered solutions (pH=4.0 to 7.4). These results indicated that the T₂ of IO-PAA-Gd-DTPA probe was not quenched upon encapsulation of Gd-DTPA complex, as hypothesized. Taken together, the above results suggest that the inverse spin-lattice magnetic relaxation (1/T₁) of the composite IO-PAA-Gd-DTPA nanoprobes got activated when exposed to acidic environments, and could be of potential use as an activatable NMR/MRI imaging agent for the detection of acidic tumors or upon internalization and localization of the nanoprobes within lysosomes.

### 1.3 Magnetic Relaxations of the Gd-DTPA Composite Nanoceria.

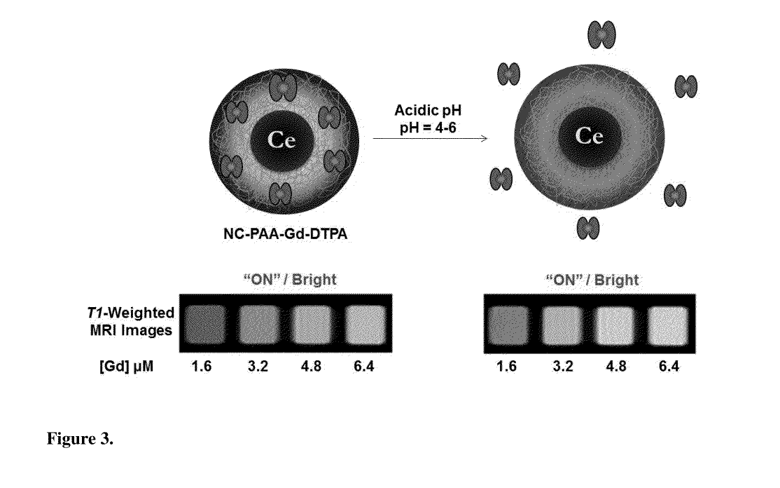

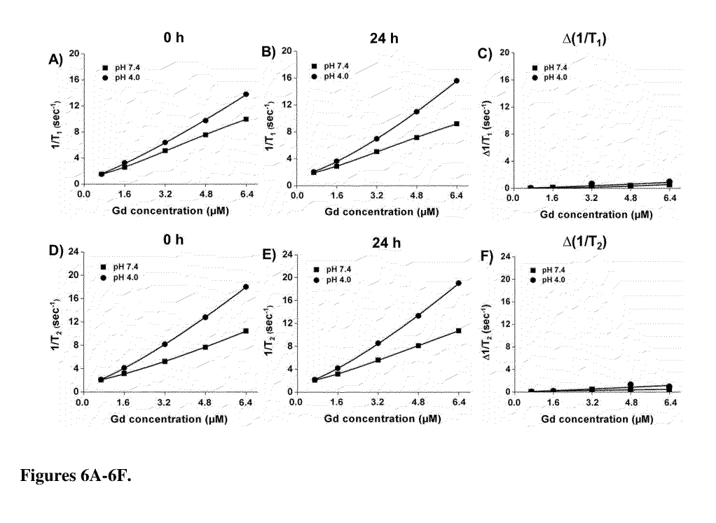

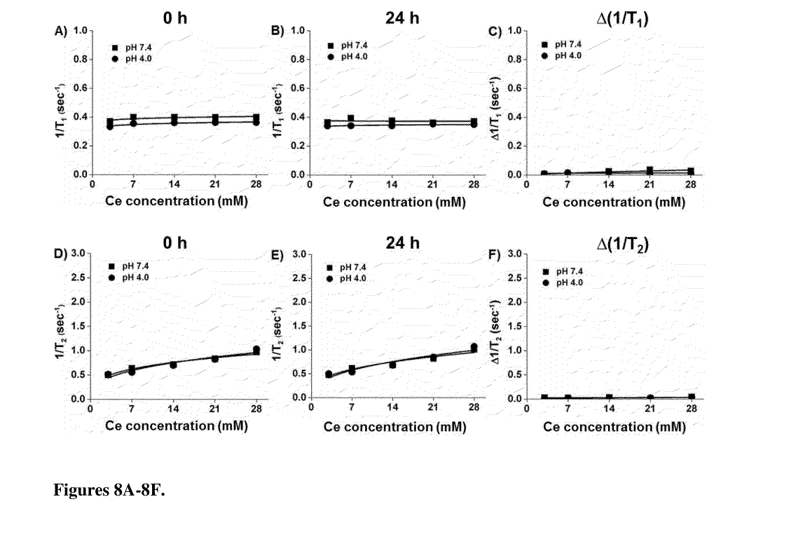

To confirm that the superparamagnetic nature of the iron oxide core is responsible for quenching the magnetic relaxation of the Gd-DTPA, a PAA coated cerium oxide nanoparticle encapsulating Gd-DTPA (NC-PAA-Gd-DTPA) was synthesized. In this design, a non-magnetic metal oxide core composed of cerium oxide (nanoceria) replaced the magnetic iron oxide core. The NC-PAA-Gd-DTPA nanoprobes were synthesized following a procedure similar to the one used to synthesize the IO-PAA-Gd-DTPA nanoprobe. Briefly, to a PAA solution in water, Gd-DTPA was added and mixed thoroughly before addition to a solution of cerium nitrate in ammonium hydroxide solutions (Scheme 3 shown in FIG. 3). The synthesized NC-PAA-Gd-DTPA composite nanoprobe was purified using the SpectrumLab's Krosflo filtration system. DLS and ICP-MS of the nanoprobe aqueous suspension indicated the presence of 88±1 nm nanoparticles with a Gd concentration of 0.315 mg/mL. These values were similar to those obtained for the IO-PAA-Gd-DTPA nanoprobes, suggesting that the size, polymer coating thickness and amount of encapsulated Gd was similar in both preparations. Magnetic relaxation values of the aqueous nanoparticle suspension revealed an R1=34.3±2.1 mM⁻¹Sec⁻¹ and R2=60±5.2 mM⁻¹Sec⁻¹ (based on Gd concentration), further confirming the successful encapsulation of Gd in the nanoparticle's polymeric core. The magnetic relaxation rates 1/T₁ and 1/T₂ of the NC-PAA-Gd-DTPA nanoprobes indicated no change in Δ1/T1 (FIG. 6C) before (0 h, FIG. 6A) or after 24 h incubation (FIG. 6B) in either physiological (pH=7.4) or acidic (pH=4.0) buffered solutions, indicating no magnetic relaxation activation at acidic pH. Similarly, no changes in T2 (Δ1/T₂, FIG. 6 D-F) were recorded for the NC-PAA-Gd-DTPA nanoprobes, and as expected no changes in magnetic relaxation rates (1/T1 and 1/T2) were observed in the case of non-magnetic nanoceria control probe NC-PAA^{61,62} (FIG. 8). Taken together, the above results suggest that the observed quenching of the Gd-DTPA T₁ relaxation rate (1/T₁) only occurred when the Gd-DTPA was encapsulated in close proximity to a superparamagnetic core (iron oxide). These data also suggest that the observed quenching is not due to immobilization of the Gd-DTPA within a polymer matrix surrounding a non-magnetic core (cerium oxide).

### 1.4 Magnetic Relaxations of the Gd-DTPA Surface Conjugating Magnetic Nanoprobes.

It was stated that the close proximity of Gd (a weak paramagnetic ion) within the polymeric matrix of iron oxide nanoparticles (a strong superparamagnetic nanocrystal) affects the T1 relaxation of Gd. If this hypothesis is correct, conjugation of a Gd-DTPA directly on the nanoparticle's surface should not result in quenching of the T₁ values. To further test this hypothesis, a IO-PAA-Gd-DTPA magnetic nanoprobe was synthesized where the Gd-DTPA was conjugated directly on the IO-PAA surface carboxylic acid groups (FIG. 7). Briefly, IO-PAA was first conjugated with ethylenediamine using the water-soluble carbodiimide chemistry, as previously described.²² The resulting aminated IO-PAA was then conjugated with Gd(III) chelated 2-(4-isothiocyanatobenzyl)-diethylenetriaminepentaacetic acid (pSCN-Bn-Gd-DTPA) in basic PBS buffer (pH=8.4). The conjugated magnetic nanoprobe was purified using small magnetic columns (Miltenyi Biotech) and washed with PBS (pH=7.4), prior to characterizations and magnetic relaxation measurements. The successful conjugation of the functional Gd-DTPA complex was confirmed by performing ICP-MS experiments and the resulting [Gd] concentration was found to be 0.201 mg/mL. The magnetic relaxation values of the conjugated nanoprobe was R₁=63.4±1.5 mM⁻¹Sec⁻¹ and R₂=92.1±3.8 mM⁻¹Sec⁻¹ (based on Gd concentration); and R1=49.9±1.3 mM⁻¹Sec⁻¹ and R2=243±3 mM⁻¹Sec⁻¹ (based on Fe concentration). The T₁ and T₂ relaxation rates (1/T₁ and 1/T₂) of the nanoprobe were measured

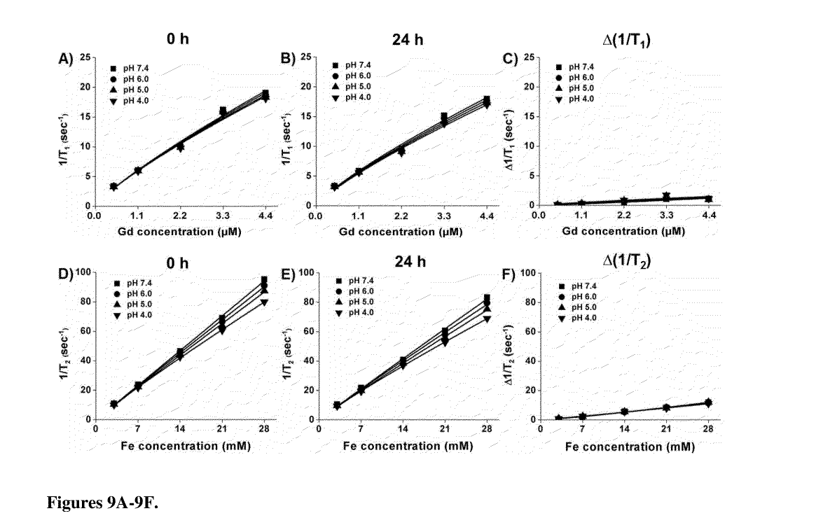

Results showed no change in Δ1/T₁ (FIG. 9C) before (0 h, FIG. 9A) or after (24 h, FIG. 9B) incubating in various buffered solutions and were found to be similar to that of control IO-PAA probe with no magnetic activation (FIGS. 5A-5F). Similarly, no changes in spin-spin relaxations (Δ1/T₂, FIG. 9D-F) were observed after the 24 h of treatment. Overall, the above results indicate that the encapsulation of the Gd-DTPA within the polymeric coating and close proximity to the iron oxide core responsible for the Gd relaxation quenching, which was then activated upon release.

Meanwhile, the R₁ and R₂ relaxation values based on Gd concentrations of the IO-PAA-Gd-DTPA nanoprobes indicate a significant pH-dependent increase in the R₁ of the nanoprobes when the Gd is encapsulated within the polymeric coating of the iron oxide nanoparticles (Table 2). Results show that by decreasing the pH of the solution to a mildly acidic condition (pH 6.0), a significant percent increase of 44% in the Gd based R₁ is observed. This value contrast with a small increase of 5% observed when the Gd is conjugated on the nanoparticle surface, further indicating that indeed encapsulation within the nanoparticle's polymeric matrix is essential for the observed T₁ activation. The observed increase in R₁ is larger at higher pH, observing a 68% increasing at pH 5.0, the typical pH within lysosomes. Even though pH-dependent percent changes in R₂ are also observed in the Gd encapsulated nanocomposite, they are not as large as the values obtained with R₁. Taken together, these results confirm the T1 activation of the IO-PAA-Gd-DTPA nanoprobes upon increases in pH, particularly within the physiological relevant range (pH 5-6) observed in tumors.

TABLE 2
Table 2. Magnetic relaxation values at 0.47 T of the nanocomposite
based on Gd concentrations at different pH.

| | | R₁ | R₂ | % Change | % Change |
|---|---|---|---|---|---|
| Nanoprobe | pH | (mM⁻¹Sec⁻¹) | (mM⁻¹Sec⁻¹) | R1 | R2 |
| IO-PAA-Gd- | 7.4 | 50.2 ± 1.8 | 87.3 ± 2.4 | — | — |
| DTPA | 6.0 | 72.5 ± 1.3 | 98.2 ± 3.2 | 44 | 12 |
| (Encapsulated) | 5.0 | 84.3 ± 1.2 | 111.6 ± 2.8 | 68 | 28 |
| [Gd] = 0.289 | 4.0 | 97.0 ± 2.5 | 118.5 ± 3.4 | 93 | 38 |
| mg/mL | | | | | |
| IO-PAA-Gd- | 7.4 | 63.4 ± 1.5 | 92.1 ± 3.8 | — | — |
| DTPA | 6.0 | 66.3 ± 2.2 | 95.2 ± 1.2 | 5 | 3 |
| (Surface) | 5.0 | 68.1 ± 1.4 | 97.4 ± 2.1 | 7 | 6 |
| [Gd] = 0.201 | 4.0 | 69.3 ± 1.3 | 98.5 ± 1.8 | 9 | 7 |
| mg/mL | | | | | |

### 1.5 MRI-Based T₁-Weighted Activation of the Composite IO-PAA-Gd-DTPA Nanoprobe.

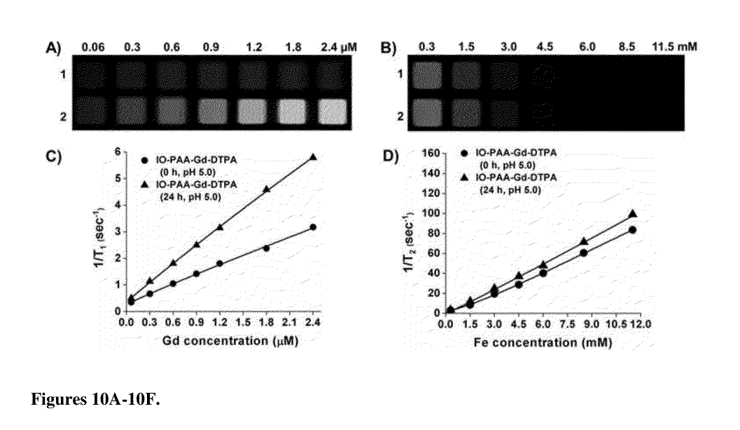

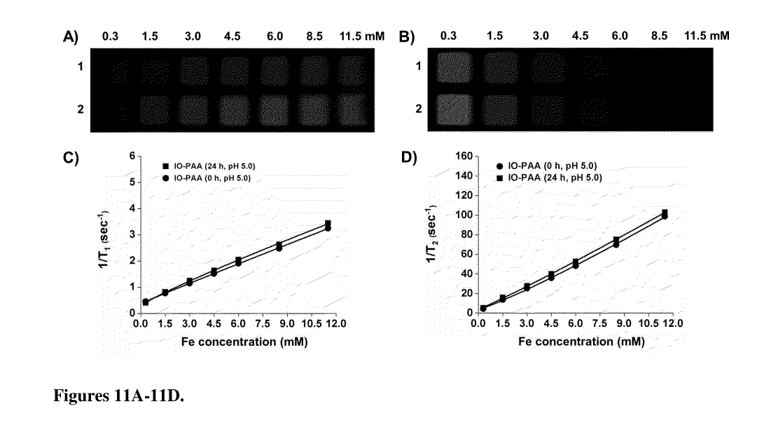

Next, it was investigated if the observed pH-dependent increases in R₁ of the IO-PAA-Gd-DTPA nanoprobe result in increases in T₁-weighted signal in MR images, leading to an increase in the brightness of the image. For these experiments, the T₁- and T₂-weighted MR images (MRI, B=4.7 T) of nanoprobe solutions at pH 5.0 were acquired immediately (FIG. 10A1) and after a 24 h incubation (FIG. 10A2) in the pH 5.0 buffer. An increase in the T₁-weighted MR signals was observed as the concentration of the activatable IO-PAA-Gd-DTPA nanoprobes increased (from 0.3 mM to 11.5 mM), resulting in an increase in the signal of the corresponding MR images (FIG. 10A2). The observed signal increase after a 24 h incubation in the pH 5.0 buffer corresponded to an increase in the (1/T₁) relaxation rate (▴, FIG. 10C). As expected, a minimal increase in T₂-weighted MR signals (T₂ Map, FIG. 10B) or corresponding inverse spin-spin magnetic relaxations (1/T₂, FIG. 10D) were observed from the IO-PAA-Gd-DTPA nanoprobes due to the absence of any T₂ activation. However, in this case the MR signals were found to be decreased, since the iron concentrations increased with the rising nanoprobe concentrations. The calculated R₁ and R₂ values at 4.7T for the IO-PAA-Gd-DTPA nanoprobe before and after a 24 h incubation at pH 5.0 also show an increase in R₁ values (24.8±1.2 vs 45.2±1.9 mM⁻¹Sec⁻¹), for a percent increase in R₁ of 87%. Meanwhile, a modest increase in R₂ was observed as (75.4±2.3 vs 91.5±3.1 mM⁻¹Sec⁻¹) for a percent increase of only 21%. In another set of experiments, no change in MR signals (both T1- and T2-Map) and corresponding magnetic relaxations were observed due to the absence of any magnetic activation from our control IO-PAA probe (FIG. 11A-11D). Taken together, the above results confirm that our activatable IO-PAA-Gd-DTPA nanoprobes get activated at acidic pH, and activation was indicated by the strong T₁-weighted MRI signals. These results also suggest the potential diagnostic applications of our novel NMR/MRI activatable composite iron oxide nanoprobes for imaging acidic tumors.

### 1.6 In Vitro Activation of the Composite IO-PAA-Gd-DTPA Nanoprobe.

To evaluate the potential biomedical applications of the activatable IO-PAA-Gd-DTPA nanoprobes, their magnetic activations were assessed using cultured cells. It was hypothesized that upon receptor mediated endocytosis, the nanoparticles will localize in acidic lysosomes, therefore becoming activated as the encapsulated Gd-DTPA complex gets released at lower pH. For these experiments, the magnetic nanoprobes were functionalized with folic acid, following published protocols,^{22,63} in order to assess their targeted imaging capabilities towards folate receptor (FR)-expressing cancer cells. It was hypothesized that upon internalization into FR expressing cancer cells, T1 activation of the composite IO-PAA-Gd-DTPA-Fol nanoprobe would be triggered by the lysosomal acidic environment (pH=5.0), resulting in vitro activation of the MRI signals. In these experiments, we used a FR positive human cervical carcinoma cell line (HeLa cells, 10,000 cells/well) and as negative control we used H9c2 cardiomyocyte (10,000 cells/well) that do not express FR. Cells were incubated with the nanoprobes (100 μL, 28 mM) at different time-points, trypsinized, centrifuged and resuspended in PBS (pH=7.4) before measuring T1 and T2 of the nanoparticle cell suspension.

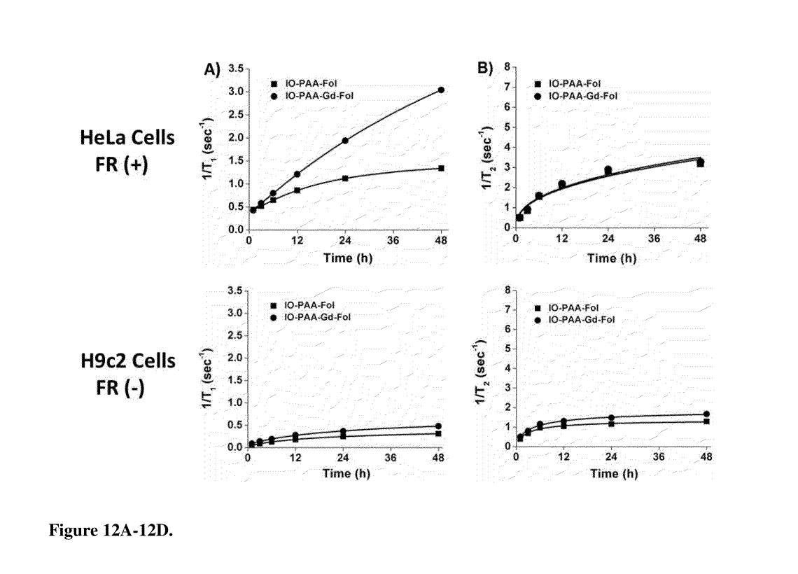

As hypothesized, compared to the control IO-PAA-Fol nanoprobes, significant activation in inverse spin-lattice magnetic relaxations (1/T_{I}) was observed in HeLa cells incubated with the activatable IO-PAA-Gd-DTPA-Fol nanoprobes (, FIG. 12A). While, no significant changes in 1/T₂ were observed from HeLa cells incubated with either of the probes (FIG. 12B). These results further supported the in vitro activatable MR imaging capability of the composite nanoprobes, whereas the control probe's (▪, IO-PAA-Fol) magnetic relaxation remained unchanged after the FR-mediated internalizations. In contrast, no significant changes in magnetic relaxations (both 1/T₁ and 1/T₂) were observed from H9c2 cells (FR negative) incubated with either one of the nanoprobes, suggested the lack of any receptor-mediated internalizations of our magnetic nanoprobes (FIGS. 12C and 12D). Taken together, the results confirmed that the FR-mediated internalizations and lysosomal acidic pH-assisted release of the encapsulating Gd-DTPA complex was responsible for the enhanced MR signal from our composite IO-PAA-Gd-DTPA-Fol nanoprobe. These results also indicated that the potential activatable MR imaging capability of synthesized composite nanoprobes could play an important role in the detection and treatment of cancer in clinical settings.

1.7 pH-Dependent Dual Release of the Gd-DTPA Complex and Taxol.

IO-PAA-Gd-DTPA nanoprobes were used to encapsulate Taxol as previously described using a solvent diffusion method.^{22,63} Briefly, to a suspension of IO-PAA-Gd-DTPA nanoprobes (2.5 mL, 28 mmol) in PBS, the dimethyl sulfoxide (DMSO) solution of Taxol (10 μL, 0.5 μg/μL) was added drop-wise at room temperature. The resulting purified IO-PAA-Gd-DTPA-Taxol nanoparticles were characterized by measuring their size using DLS (D=84±2 nm), taxol encapsulation efficiency (EE)=52±2.4% using HPLC (λ_{abs}=227 nm) and calculating the Gd concentration (0.215 mg/mL) by performing ICP-MS experiments. To evaluate the dual release of Taxol and Gd, the IO-PAA-Gd-DTPA-Taxol nanoprobe were incubated in a pH 5.0 buffered PBS solutions and the rate of release of the drug and Gd was accessed using a dynamic dialysis technique. Briefly, the IO-PAA-Gd-DTPA-Taxol nanoprobes (50 μL, 28 mM) were taken in a small dialysis cup (MWCO 6-8 KDa) and incubated in PBS buffer (pH=5.0) solution at 37° C. The rate of release of taxol and Gd was monitored by collecting aliquots from the outside reservoir buffer and measuring the amount of released taxol via HPLC experiment (λ_{abs}=227 nm) and Gd by measuring the increase in T1 relaxation rate with time.

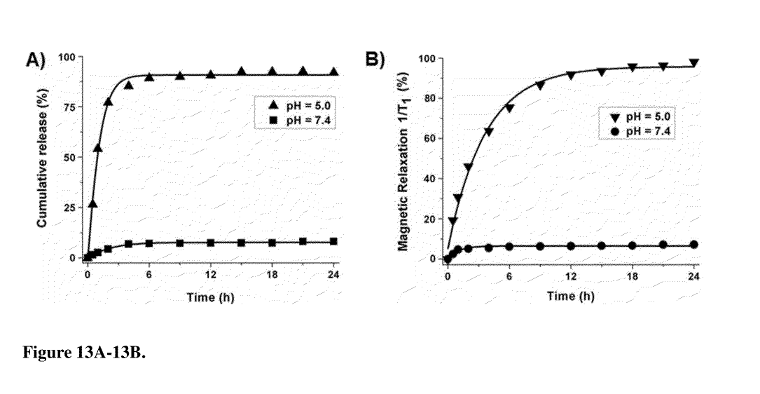

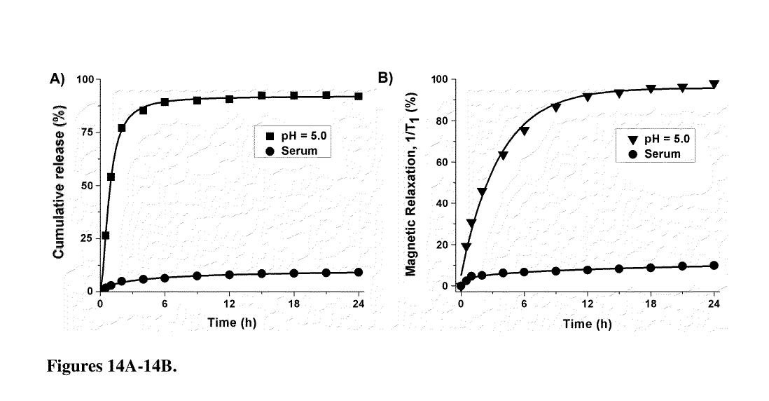

Results showed a time dependent increase in the amount of Taxol (▴, FIG. 13A) and Gd-DTPA (▾, FIG. 13B) released upon incubation at pH 5.0. These results suggest that indeed the acid-mediated degradation and/or swelling of the PAA coatings results in the simultaneous release of both Taxol and Gd. Interestingly, a slower rate of Gd-DTPA release from the nanoprobe is observed in contrast to Taxol, this could be due to a possible higher extend of H-bonding between Gd-DTPA and the carboxylic groups within the polymeric coating internal cavities surrounding the iron oxide core. In contrast, when similar experiments were performed at physiological pH (PBS, pH=7.4, 37° C.), no significant release of Taxol (▪, FIG. 13A) or Gd was observed (, FIG. 13B). Similarly, no significant release of Taxol or increase in magnetic relaxations (1/T_{I}) was observed when the IO-PAA-Gd-DTPA-Taxol nanoprobe was incubated in serum at 37° C. (FIGS. 14A-4B). These findings indicate that the IO-PAA-Gd-DTPA-Taxol nanoprobe is stable at neutral pH and physiological conditions, only releasing its cargo (Taxol and Gd) in an acidic environment.

### 1.8 In Vitro Cytotoxicity of Taxol-Encapsulating Activatable IO-PAA-Gd-DTPA Nanoprobes.

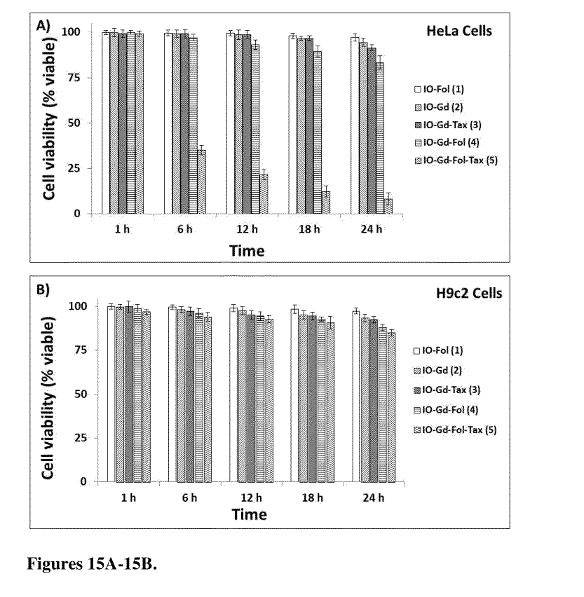

Finally, the differential in vitro cytotoxicity of the functionalized magnetic nanoprobes (35 μL, 28 mM in PBS pH=7.4) was examined using FR expressing human cervical cancer cells (HeLa, 2500 cells/well) and FR negative cardiomyocyte cell lines (H9c2, 2500 cells/well). Results confirmed a time-dependent decrease in the number of viable HeLa cells, when incubated with folate-decorated IO-PAA-Gd-DTPA-Taxol nanoprobes 5 (FIG. 15A), showing more than 90% reduction in cell viability after 24 h of incubation. However, the folate-decorated IO-PAA-Gd-DTPA nanoprobes showed nominal toxicity (4) and comparable with the IO-PAA-Fol (1) lacking Gd-DTPA complex, as published earlier.²² As expected, nominal cytotoxicity was observed when HeLa cells were incubated with the IO-PAA-Gd-DTPA (2) and IO-PAA-Gd-DTPA-Taxol (3), due to absence of any receptor-mediated internalizations. These results suggest that the cytotoxicity of the nanoprobes was not affected by the encapsulation of Gd due to the presence of PAA polymer coatings. In addition, no significant reduction in cell viability was observed when H9c2 cells, which do not overexpress FR, were incubated with all the functional magnetic nanoprobes (FIG. 15B), suggesting biocompatibility and potential applications of our nanoprobes for the targeted imaging and treatment of cancers. Taken together, the above results suggest that our folate-decorated activatable IONP-PAA-Gd-DTPA-Taxol nanoprobe can detect tumors using MR imaging, while target and deliver chemotherapeutic agents taxol using folate receptors.

In one aspect, a novel activatable Gd-DTPA-encapsulating iron oxide NMR/MRI probe is reported where the longitudinal (spin-lattice) magnetic relaxation (T₁) of the encapsulated Gd-DTPA was quenched (low 1/T₁) by the iron oxide nanoparticles (IONP-PAA). The above results clearly indicated that the magnetic relaxation of Gd-DTPA complex (T₁ agent) is quenched as a result of such encapsulation, whereas the transverse (spin-spin) magnetic relaxation (T₂) of iron oxide had a minimal increase. The T₁ relaxation of the Gd-DTPA complex becomes activated (dequenched, higher 1/T₁) and the corresponding enhanced contrast in T₁-weighted MRI experiments is observed upon acid-mediated degradation and release of the T₁ agent. In addition, it was confirmed that the T₂ activation of the probes was not quenched upon encapsulation of Gd-DTPA complex.

The results also demonstrated that the folate receptor-mediated internalization and the subsequent lysosomal acidic pH-induced intracellular release of Gd-DTPA complex resulted in an enhanced 1/T₁ signal. In addition, when the taxol-encapsulating activatable magnetic nanoprobes are incubated, the drug's homing was monitored through an enhanced MRI signal, as further confirmed in the cytotoxicity assays. The presence of folate on the activatable magnetic nanoprobe guarantees a selective activation and release of drug only in folate-receptor positive cells, minimizing toxicity to healthy cells. In contrast, no T₁ activation is observed in Gd-DTPA surface conjugating IONPs or Gd-DTPA encapsulating non-magnetic NC-PAA, confirming that quenching was due to the close residence of the Gd-DTPA to the superparamagnetic iron oxide (IO) core and not due to the presence of any non-magnetic metallic core (cerium oxide) or polymeric (PAA) coatings. Finally, the excellent physiological and plasma stability of the designed activatable and theranostic NMR/MRI probes may play an important role for the detection and treatment of cancer in clinical settings.

### 2. Materials and Methods

### 2.1 Materials.

Iron salts: ferrous(II) chloride tetrahydrate (FeCl₂.4H₂O) and ferric(III) chloride hexahydrate (FeCl₃.6H₂O), gadolinium(III) chloride hexahydrate (GdCl₃.6H₂O), cerium(III) nitrate hexahydrate (CeNO₃.6H₂O), diethylenetriaminepentaacetic acid (DTPA), ammonium hydroxide, hydrochloric acid, sodium hydroxide, chloropropryl amine, sodium azide, copper(I) iodide, ethylenediamine (EDA), folic acid, N,N-dimethylformamide (DMF), dimethyl sulfoxide (DMSO), 3-(4,5-dimethylthiazol-2-yl)-2,5-diphenyltetrazolium bromide (MTT), N-hy droxysuccinimide (NHS), 2-(N-morpholino) ethanesulfonic acid (MES), polyacrylic acid (PAA) and other chemicals were purchased from Sigma-Aldrich. 2-(4-isothiocyanatobenzyl)-diethylenetriaminepentaacetic acid [p-SCN-Bn-DTPA] was purchased from Macrocyclics. EDC [1-Ethyl-3-(3-dimethylaminopropyl) carbodiimide hydrochloride] was obtained from Pierce Biotechnology. The human cervical carcinoma (HeLa) and cardiomyocyte (H9c2) cell lines were obtained from ATCC. Magnetic columns (LS Column) were purchased from Miltenyi Biotech for the purification of magnetic nanoprobes using QuadroMACS separators. Dialysis membranes were obtained from Spectrum Laboratories. Nitrogen purged DI water was used in all synthesis.

### 2.2 Synthesis of the Gd-DTPA Complexes.

Chelation of the rare-earth element Gadolinium (Gd) with diethylenetriaminepentaacetic acid (DTPA) or with functional DTPA, p-SCN-Bn-DTPA [2-(4-isothiocyanatobenzyl)-diethylenetriaminepentaacetic acid] results in a strongly paramagnetic, stable complex that is well tolerated in animals. These complexes were synthesized following the literature reported method.^{64,65} Briefly, a solution of GdCl₃.6H₂O (4.49 g, 0.0121 mol) in H₂O (10 mL) was added drop-wise to a solution of DTPA (5.0 g, 0.0127 mol) or p-SCN-Bn-DTPA (0.0127 mol) in H₂O (30 mL) containing 2N NaOH (5.0 mL) solution. The pH of the final reaction mixture was maintained at pH 6.8 by constant addition of 2N NaOH solution. The reaction was continued at 80° C. for 12 h before concentrated to 20 mL. The observed white crystals were dissolved in minimum amount of water before precipitating in ethanol. The precipitate was filtered and dried under vacuum to obtain the Gd(III) complex as a white solid (Yield: 86%).

### 2.3 Synthesis of the Gd-DTPA-Encapsulating Composite Iron Oxide Nanoprobes (IO-PAA-Gd-DTPA).

For the synthesis of Gd-DTPA-encapsulating composite nanoprobe (IO-PAA-Gd-DTPA), a novel water-based, ‘in situ’ encapsulation approach was used for the successful encapsulation of Gd-DTPA complex. In this approach, three different solutions were prepared; an iron salt solution [0.61 g of FeCl₃. 6H₂O and 0.32 g of FeCl₂. 4H₂O in dilute HCl solution (100 μL of 12 N HCl in 2.0 mL H₂O)]; an alkaline solution [1.8 mL of 30% NH₄OH solution in 15 mL of N₂ purged DI water]; and a paramagnetic stabilizing solution [800 mg of PAA and 20 mg of Gd-DTPA complex in 5 mL of DI water]. To synthesize the composite IO-PAA-Gd-DTPA nanoprobe, the iron salt solution was added to the alkaline solution under vigorous stirring. The resulting dark suspension of iron oxide nanoparticles was stirred for 10 seconds before addition of the paramagnetic stabilizing solution and stirred for 1 h. The resulting suspension of composite IO-PAA-Gd-DTPA nanoprobe was then centrifuged at 4000 rpm for 30 minutes to get rid of free polyacrylic acid, Gd-DTPA complex and other unreacted reagents. Finally, the composite IO-PAA-Gd-DTPA nanoprobe suspension was purified using magnetic columns and washed with phosphate buffer saline (pH=7.4) solution. The iron concentration and magnetic relaxation of the PAA-IONPs was determined as previously reported.²² The successful coating of the IONPs with PAA was confirmed by the presence of a negative zeta-potential (ζ=−41 mV) and the characteristic acid carbonyl band on the FT-IR spectroscopic analysis of the nanoparticles (FIG. 2).

### 2.4 Synthesis of the Theranostic Cargos-Encapsulating Composite Activatable Magnetic Nanoprobes.

Taxol was encapsulated in the PAA polymer coating of magnetic nanoprobe, following the previously reported solvent diffusion method.^{22,66} Briefly, to a suspension of IO-PAA-Gd-DTPA nanoprobes (2.5 mL, 28 mmol) in PBS, a dimethyl sulfoxide (DMSO) solution of Taxol (10 μL, 0.5 μg/μL) was added drop-wise at room temperature with continuous stirring at 1000 rpm. The taxol-encapsulating nanoprobes (IO-PAA-Gd-DTPA-Taxol) were purified using magnetic column (Miltenyi Biotech) and then dialyzed (using 6-8K MWCO dialysis bag) three times against deionized water and finally against phosphate buffered saline solution. The resulting IO-PAA-Gd-DTPA-Taxol nanoparticles were characterized by measuring their size using DLS (D=84±2 nm), the taxol encapsulation efficiency (EE)=52±2.4% using HPLC (λ_{abs}=227 nm).

### 2.5 Synthesis of Folate-Decorated Magnetic Nanoprobes: Click Chemistry.

To synthesize folate-decorated functional IO-PAA nanoprobes, the surface carboxylic acid groups of the nanoprobes were alkynated using propargylamine as a reagent and the water-based carbodiimide chemistry was followed as previously reported.²² The resulting alkynated IO-PAA nanoprobes were purified using magnetic columns. The highly specific “click” chemistry was used to conjugate an azide-functionalized folic acid with the purified alkynated IO-PAA, as described in the previously reported methods.^{22,67} Briefly, the alkynated IO-PAA (4.0×10⁻³ mmol) in bicarbonate buffer (pH=8.5) were taken to an eppendorf tube containing catalytic amount of CuI (5.0×10⁻¹⁰ mmol) in 250 μL of bicarbonate buffer (pH=8.5) and vortexed. To the resulting solution, the azide-functionalized folic acid^{22,63} (8.0×10⁻² mmol) in DMSO was added and the reaction was incubated at room temperature for 12 h. The synthesized folate-decorated IO-PAA was purified using the magnetic column and finally washed using PBS solution (pH=7.4). The folate-decorated IO-PAA was stored in refrigerator for further characterization.

### 2.6 Synthesis of the Gd-DTPA-Encapsulating Composite Nanoceria (NC-PAA-Gd-DTPA).

For the synthesis of Gd-DTPA-encapsulating composite nanoceria, we have modified our previously reported stepwise method^{61,68} and followed the ‘in situ’ encapsulation approach. In this approach, 1M cerium(III) nitrate (2.17 g in 5.0 mL of water) solution was added to 30.0 mL of ammonium hydroxide solution (30% w/v) under continuous stirring at room temperature. Then, after 45 seconds of stirring, an aqueous mixture containing the PAA polymer and Gd-DTPA complex (800 mg of PAA and 20 mg of Gd-DTPA in 5 mL of water) was added and allowed to stir for 3 h at room temperature. The preparation was then centrifuged at 4000 rpm for two 30 minute cycles to settle down any debris and large agglomerates. The supernatant solution was then purified from free PAA, Gd-DTPA complex or other chemicals and concentrated using SpectrumLab's KrosFlo filtration system.

### 2.7 Synthesis of the Gd-DTPA Surface Conjugating Magnetic Nanoprobes (IO-PAA-Gd-DTPA-Surface).

The polyacrylic acid coated iron oxide nanoparticles (IO-PAA) were synthesized using our previously reported alkaline precipitation method.²² Briefly, a Fe⁺³/Fe⁺² solution in water was rapidly mixed with an ammonium hydroxide solution for 30 seconds, prior to addition of the PAA polymer solution in water. The synthesized IO-PAA were purified using magnetic columns to remove any unreacted reagents and phosphate buffered saline (PBS, pH 7.4) was used as running solvent. To incorporate amine groups to the nanoparticles, ethylenediamine was used as an aminating agent and the water-based carbodiimide chemistry (using EDC and NHS reagents) was followed, as previously reported.^{22,62} The successful amination of the IO-PAA nanoparticles were confirmed by measuring their overall positive surface charge (zeta potential ζ=+15 mV) using Malvern's Zetasizer. To synthesize the Gd-DTPA surface conjugating IO-PAA nanoprobe, the aminated IO-PAA was reacted with the isothiocyanate group of the p-SCN-Bn-DTPA chelated with GdCl₃.6H₂O salt. In a typical reaction, the isothiocyanate functional Gd-DTPA chelate (pSCN-Bn-Gd-DTPA, 25 mmol) was added to the aminated IO-PAA nanoprobe (1 mmol) in the presence of basic phosphate buffered saline (PBS, pH 8.4) and incubated overnight at room temperature. The resulting Gd-DTPA surface conjugating IO-PAA nanoprobe was purified using small magnetic columns (Miltenyi Biotech) and washed with phosphate buffered saline (PBS, pH=7.4), prior to characterizations and magnetic relaxation measurements.

### 2.8 Measurement of the Hydrodynamic Diameter and Surface Zeta Potential of the Functional IO-PAA.

The size and dispersity of the synthesized composite IO-PAA was measured using dynamic light scattering (DLS) using PDDLS/CoolBatch 40T instrument with Precision Deconvolve 32 software. The overall surface charges (zeta potential) of this functional IO-PAA were measured using a Zetasizer Nano ZS from Malvern Instruments. These experiments were performed by placing 10 μL of the composite magnetic nanoprobes in 990 μL of distilled water.

### 2.9 Measurement of Magnetic Relaxations.

Magnetic relaxation measurements were conducted with a compact magnetic relaxometer (0.47 T mq20, Bruker), by taking composite magnetic nanoprobes at the end of the experiment. Magnetic resonance imaging (MRI) of the magnetic phantoms was achieved using the MRI/MRS facility utilizing a 4.7-T 33-cm bore magnet imaging/spectroscopy system (MSKCC, New York).

### 2.10 HPLC Experiment.

HPLC experiments were carried out using PerkinElmer's Series 200 instrument to study drug release kinetics. In a typical experiment, upon addition of acidic PBS solution (pH=5.0) to the taxol-encapsulating IO-PAA-Gd-DTPA (50 μL, 28 mM), the rate of release of encapsulating taxol was monitored in a timely manner at 37° C. using HPLC (λ_{abs}=227 nm) chromatography.

### 2.11 Cell Cultures.

The human cervical cancer (HeLa) and cardiomyocyte (H9c2) cells were obtained from ATCC, and maintained in accordance to the supplier's protocols. Briefly, the cervical cancer cells were grown in a 5%-FBS-containing DMEM medium supplemented with L-glutamine, streptomycin, amphotericin B and sodium bicarbonate. The H9c2 cells were propagated in a 10% FBS-containing MEM medium containing penicillin, streptomycin and bovine insulin (0.01 mg/mL). Cells were grown in a humidified incubator at 37° C. under 5% CO₂ atmosphere.

### 2.12 In Vitro Magnetic Activations of the Composite Nanoprobes.

The human cells (HeLa and H9c2, 10,000 cells/well) were incubated with the folate-decorated activatable IO-PAA-Gd-DTPA-Fol nanoprobe and the control IO-PAA-Fol nanoprobe (100 μL, 28 mM) at different incubation times. The cells were then trypsinized and centrifuged. The resulting cell pellet was suspended in phosphate buffer saline (PBS, pH=7.4) and magnetic relaxations of these solutions were measured using the bench-top magnetic relaxometer (B=0.47 T mq 20) from Bruker.

### 2.13 Cytotoxicity Assay.

H9c2 and HeLa cells (2,500 cells/well) were seeded in 96-well plates, incubated with the corresponding composite IO-PAA nanoprobes (35 μL, 28 mM in PBS pH=7.4) at 37° C. After the specific time incubation, each well was washed three times with 1×PBS and treated with 30 μL MTT (2 μg/μL) for 2 h. The resulting formazan crystals were dissolved in acidic isopropanol (0.1 N HCl) and the absorbance was recorded at 570 and 750 nm (background), using a Synergy μQuant microtiter plate reader (Biotek). These experiments were performed in triplicates.

### 2.14 Supporting Information Available:

Detailed physical characterizations of the magnetic probes including dynamic light scattering (DLS), scanning transmittance electron microscopy (STEM), FT-IR, zeta potential, stability of the nanoprobes at different conditions, MRI-based magnetic relaxations, encapsulating drug release and cytotoxicity studies. This material is available free of charge via the Internet at http://pubs.acs.org.

### 3.1 Gd and Doxorubicin Conjugate.

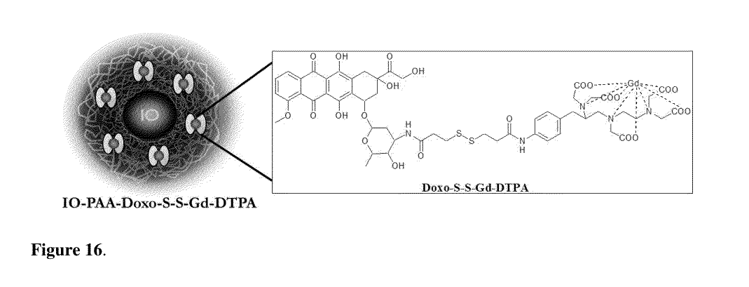

As shown in FIG. 16, Gd and Doxorubicin were attached via a disulfide bond creating a conjugate that can be encapsulated in the polymeric coating of the iron oxide nanoparticles. Note that in the examples provided above, the Gd and the anti-cancer agent were co-encapsulated as unique entities as opposed to a conjugated entity. Either way, similar results are observed when either the Doxorubicin and Gd are provided as separate entities or when Doxorubicin-ss-Gd(DTPA) are released from the nanocomposite at acidic pH. In both cases, a significant activation in the R₁ is obtained.

It was found that using a Doxorubicin-ss-Gd(DTPA), the effect on R₂ is not as large as the effect on R₁. Similar results were obtained by co-encapsulation Gd(DTPA) and Doxorubicin, although the changes are lower. This fact is significant as the release of the drug from the nanoparticle will result in activation of the probe with potential imaging monitoring of the drug release by MRI.

TABLE 3
Table 3: Change in magnetic relaxations (% change)
R₁ and R₂ based on Gd and Fe concentrations respectively,
using 0.47 T magnetic relaxometer at different pH.

| | | | % change |
|---|---|---|---|
| pH | R₁ (0 h) | R₁ (24 h) | R₁ [Gd] |
| 7.4 | 52.4 | 53.1 | 1 |
| 6.0 | 55.1 | 89.6 | 62 |
| 5.0 | 56.5 | 110.3 | 95 |
| 4.0 | 56.8 | 137.8 | 142 |
| | | | % change |
| pH | R₂ (0 h) | R₂ (24 h) | R₂ [Fe] |
| 7.4 | 232.3 | 240.0 | 3 |
| 6.0 | 255.5 | 340.7 | 33 |
| 5.0 | 263.2 | 402.6 | 53 |
| 4.0 | 271.0 | 449.1 | 65 |

The cleavable “Doxo-S-S-Gd-DTPA” conjugate were synthesized using a facile nucleophilic substitution reaction before encapsulating with IO-PAA as described earlier in the case of IO-PAA-Gd-DTPA. In a typical reaction, the aqueous solution of doxorubicin hydrochloride salt (1.75 mmol) was added to PBS buffer solution (pH=8.4) to obtain doxorubicin with free amine group. The resulting solution was centrifuged and the solid pallet was soluble in DMSO. Then, p-NH₂-Bn-Gd-DTPA complex (1.75 mmol, in PBS, pH 7.4) and dithiobis(succinimidyl propionate) (DSP) solution (1.75 mmol, in DMSO) were added drop-wise. A catalytic amount of triethylamine (0.5 μL in DMSO) was added to the reaction mixture. The reaction mixture was incubated at room temperature for 30 minutes, before overnight incubation at 4° C. (FIG. 7). The final product “Doxo-S-S-Gd-DTPA” was purified following chromatographic methods and kept at 4° C. as stock solution.

### 4. REFERENCES

(1) Winter, P. M.; Caruthers, S. D.; Wickline, S. A.; Lanza, G. M. Molecular imaging by MRI. Curr. Cardio. Reports 2006, 8, 65-69.
(2) Hu, X.; Norris, D. G. Advances in high-field magnetic resonance imaging. Ann. Rev. Biomed. Eng. 2004, 6, 157-184.
(3) Aime, S.; Cabella, C.; Colombatto, S.; Geninatti Crich, S.; Gianolio, E.; Maggioni, F. Insights into the use of paramagnetic Gd(III) complexes in MR-molecular imaging investigations. J. Magn. Reson. Imaging 2002, 16, 394-406.
(4) Louie, A. Multimodality imaging probes: design and challenges. Chem. Rev. 2010, 110, 3146-3195.
(5) Mulder, W. J.; Strijkers, G. J.; Griffioen, A. W.; van Bloois, L.; Molema, G.; Storm, G.; Koning, G. A.; Nicolay, K. A liposomal system for contrast-enhanced magnetic resonance imaging of molecular targets. Bioconjugate Chem. 2004, 15, 799-806.
(6) Mazooz, G.; Mehlman, T.; Lai, T. S.; Greenberg, C. S.; Dewhirst, M. W.; Neeman, M. Development of magnetic resonance imaging contrast material for in vivo mapping of tissue transglutaminase activity. Cancer Res. 2005, 65, 1369-1375.
(7) Josephson, L.; Tung, C. H.; Moore, A.; Weissleder, R. High-efficiency intracellular magnetic labeling with novel superparamagnetic-Tat peptide conjugates. Bioconjugate Chem. 1999, 10, 186-191.
(8) Caravan, P.; Ellison, J. J.; McMurry, T. J.; Lauffer, R. B. Gadolinium(III) Chelates as MRI Contrast Agents: Structure, Dynamics, and Applications. Chem. Rev. 1999, 99, 2293-2352.
(9) Song, Y.; Zong, H.; Trivedi, E. R.; Vesper, B. J.; Waters, E. A.; Barrett, A. G.; Radosevich, J. A.; Hoffman, B. M.; Meade, T. J. Synthesis and characterization of new porphyrazine-Gd(III) conjugates as multimodal MR contrast agents. Bioconjugate Chem. 2010, 21, 2267-2275.
(10) Frullano, L.; Meade, T. J. Multimodal MRI contrast agents. J. Biol. Iinorg. Chem. 2007, 12, 939-949.
(11) Tu, C. Q.; Louie, A. Y. Photochromically-controlled, reversibly-activated MRI and optical contrast agent. Chem. Commun. 2007, 1331-1333.
(12) Uzgiris, E. E.; Cline, H.; Moasser, B.; Grimmond, B.; Amaratunga, M.; Smith, J. F.; Goddard, G. Conformation and structure of polymeric contrast agents for medical imaging. Biomacromolecules 2004, 5, 54-61.
(13) Endres, P. J.; MacRenaris, K. W.; Vogt, S.; Meade, T. J. Cell-permeable MR contrast agents with increased intracellular retention. Bioconjugate Chem. 2008, 19, 2049-2059.
(14) Laurent, S.; Forge, D.; Port, M.; Roch, A.; Robic, C.; Vander Elst, L.; Muller, R. N. Magnetic iron oxide nanoparticles: synthesis, stabilization, vectorization, physicochemical characterizations, and biological applications. Chem. Rev. 2008, 108, 2064-2110.
(15) Hu, F.; Macrenaris, K. W.; Waters, E. A.; Schultz-Sikma, E. A.; Eckermann, A. L.; Meade, T. J. Highly dispersible, superparamagnetic magnetite nanoflowers for magnetic resonance imaging. Chem. Commun. 2010, 46, 73-75.
(16) Cho, S. J.; Jarrett, B. R.; Louie, A. Y.; Kauzlarich, S. M. Gold-coated iron nanoparticles: a novel magnetic resonance agent for T-1 and T-2 weighted imaging. Nanotechnology 2006, 17, 640-644.
(17) Huber, M. M.; Staubli, A. B.; Kustedjo, K.; Gray, M. H.; Shih, J.; Fraser, S. E.; Jacobs, R. E.; Meade, T. J. Fluorescently detectable magnetic resonance imaging agents. Bioconjugate Chem. 1998, 9, 242-249.
(18) Hooker, J. M.; Datta, A.; Botta, M.; Raymond, K. N.; Francis, M. B. Magnetic resonance contrast agents from viral capsid shells: a comparison of exterior and interior cargo strategies. Nano Lett. 2007, 7, 2207-2210.
(19) Crich, S. G.; Biancone, L.; Cantaluppi, V.; Duo, D.; Esposito, G.; Russo, S.; Camussi, G.; Aime, S. Improved route for the visualization of stem cells labeled with a Gd-/Eu-chelate as dual (MRI and fluorescence) agent. Magn. Reson. Med. 2004, 51, 938-944.
(20) Brekke, C.; Morgan, S. C.; Lowe, A. S.; Meade, T. J.; Price, J.; Williams, S. C.; Modo, M. The in vitro effects of a bimodal contrast agent on cellular functions and relaxometry. NMR in Biomed. 2007, 20, 77-89.
(21) Anderson, E. A.; Isaacman, S.; Peabody, D. S.; Wang, E. Y.; Canary, J. W.; Kirshenbaum, K. Viral nanoparticles donning a paramagnetic coat: conjugation of MRI contrast agents to the MS2 capsid. Nano Lett. 2006, 6, 1160-1164.
(22) Santra, S.; Kaittanis, C.; Grimm, J.; Perez, J. M. Drug/dye-loaded, multifunctional iron oxide nanoparticles for combined targeted cancer therapy and dual optical/magnetic resonance imaging. Small 2009, 5, 1862-1868.
(23) Chen, T.; Shukoor, M. I.; Wang, R.; Zhao, Z.; Yuan, Q.; Bamrungsap, S.; Xiong, X.; Tan, W. Smart multifunctional nanostructure for targeted cancer chemotherapy and magnetic resonance imaging. ACS Nano 2011, 5, 7866-7873.
(24) Kim, H. M.; Lee, H.; Hong, K. S.; Cho, M. Y.; Sung, M. H.; Poo, H.; Lim, Y. T. Synthesis and high performance of magnetofluorescent polyelectrolyte nanocomposites as MR/near-infrared multimodal cellular imaging nanoprobes. ACS Nano 2011, 5, 8230-8240.
(25) Paquet, C.; de Haan, H. W.; Leek, D. M.; Lin, H. Y.; Xiang, B.; Tian, G.; Kell, A.; Simard, B. Clusters of superparamagnetic iron oxide nanoparticles encapsulated in a hydrogel: a particle architecture generating a synergistic enhancement of the T2 relaxation. ACS Nano 2011, 5, 3104-3112.
(26) Poselt, E.; Kloust, H.; Tromsdorf, U.; Janschel, M.; Hahn, C.; Masslo, C.; Weller, H. Relaxivity Optimization of a PEGylated Iron-Oxide-Based Negative Magnetic Resonance Contrast Agent for T(2)-Weighted Spin-Echo Imaging. ACS Nano 2012, 6, 1619-1624.
(27) Tromsdorf, U. I.; Bigall, N. C.; Kaul, M. G.; Bruns, O. T.; Nikolic, M. S.; Mollwitz, B.; Sperling, R. A.; Reimer, R.; Hohenberg, H.; Parak, W. J.; Forster, S.; Beisiegel, U.; Adam, G.; Weller, H. Size and surface effects on the MRI relaxivity of manganese ferrite nanoparticle contrast agents. Nano Lett. 2007, 7, 2422-2427.
(28) Tu, C.; Ng, T. S.; Sohi, H. K.; Palko, H. A.; House, A.; Jacobs, R. E.; Louie, A. Y. Receptor-targeted iron oxide nanoparticles for molecular MR imaging of inflamed atherosclerotic plaques. Biomaterials 2011, 32, 7209-7216.
(29) Bae, K. H.; Kim, Y. B.; Lee, Y.; Hwang, J.; Park, H.; Park, T. G. Bioinspired Synthesis and Characterization of Gadolinium-Labeled Magnetite Nanoparticles for Dual Contrast T(1)- and T(2)-Weighted Magnetic Resonance Imaging. Bioconjugate Chem. 2010, 21, 505-512.
(30) Bull, S. R.; Guler, M. O.; Bras, R. E.; Meade, T. J.; Stupp, S. I. Self-assembled peptide amphiphile nanofibers conjugated to MRI contrast agents. Nano Lett. 2005, 5, 1-4.
(31) Bull, S. R.; Guler, M. O.; Bras, R. E.; Venkatasubramanian, P. N.; Stupp, S. I.; Meade, T. J. Magnetic resonance imaging of self-assembled biomaterial scaffolds. Bioconjugate Chem. 2005, 16, 1343-1348.
(32) Datta, A.; Hooker, J. M.; Botta, M.; Francis, M. B.; Aime, S.; Raymond, K. N. High relaxivity gadolinium hydroxypyridonate-viral capsid conjugates: nanosized MRI contrast agents. J. Am. Chem. Soc. 2008, 130, 2546-2552.
(33) Drake, P.; Cho, H. J.; Shih, P. S.; Kao, C. H.; Lee, K. F.; Kuo, C. H.; Lin, X. Z.; Lin, Y. J. Gd-doped iron-oxide nanoparticles for tumour therapy via magnetic field hyperthermia. J. Mater. Chem. 2007, 17, 4914-4918.
(34) Pan, D.; Caruthers, S. D.; Hu, G.; Senpan, A.; Scott, M. J.; Gaffney, P. J.; Wickline, S. A.; Lanza, G. M. Ligand-directed nanobialys as theranostic agent for drug delivery and manganese-based magnetic resonance imaging of vascular targets. J. Am. Chem. Soc. 2008, 130, 9186-9187.
(35) Song, Y.; Kohlmeir, E. K.; Meade, T. J. Synthesis of multimeric MR contrast agents for cellular imaging. J. Am. Chem. Soc. 2008, 130, 6662-6663.
(36) Song, Y.; Xu, X.; MacRenaris, K. W.; Zhang, X. Q.; Mirkin, C. A.; Meade, T. J. Multimodal gadolinium-enriched DNA-gold nanoparticle conjugates for cellular imaging. Angew. Chem. Int. Ed. Engl. 2009, 48, 9143-9147.
(37) Cheng, Z.; Thorek, D. L.; Tsourkas, A. Gadolinium-conjugated dendrimer nanoclusters as a tumor targeted T1 magnetic resonance imaging contrast agent. Angew. Chem. Int. Ed. Engl. 2010, 49, 346-350.
(38) Cheng, Z. L.; Thorek, D. L. J.; Tsourkas, A. Porous polymersomes with encapsulated Gd-labeled dendrimers as highly efficient MRI contrast agents. Adv. Funct. Mater. 2009, 19, 3753-3759.
(39) Allen, M. J.; MacRenaris, K. W.; Venkatasubramanian, P. N.; Meade, T. J. Cellular delivery of MRI contrast agents. Chemistry & Biology 2004, 11, 301-307.
(40) Manus, L. M.; Mastarone, D. J.; Waters, E. A.; Zhang, X. Q.; Schultz-Sikma, E. A.; Macrenaris, K. W.; Ho, D.; Meade, T. J. Gd(III)-nanodiamond conjugates for MRI contrast enhancement. Nano Lett. 2010, 10, 484-489.
(41) Mastarone, D. J.; Harrison, V. S.; Eckermann, A. L.; Parigi, G.; Luchinat, C.; Meade, T. J. A modular system for the synthesis of multiplexed magnetic resonance probes. J. Am. Chem. Soc. 2011, 133, 5329-5337.
(42) Major, J. L.; Meade, T. J. Bioresponsive, cell-penetrating, and multimeric MR contrast agents. Acc. Chem. Res. 2009, 42, 893-903.
(43) Kalman, F. K.; Woods, M.; Caravan, P.; Jurek, P.; Spiller, M.; Tircso, G.; Kiraly, R.; Brucher, E.; Sherry, A. D. Potentiometric and relaxometric properties of a gadolinium-based MRI contrast agent for sensing tissue pH. Inorg. Chem. 2007, 46, 5260-5270.
(44) Duimstra, J. A.; Femia, F. J.; Meade, T. J. A gadolinium chelate for detection of beta-glucuronidase: a self-immolative approach. J. Am. Chem. Soc. 2005, 127, 12847-12855.
(45) Caravan, P.; Cloutier, N. J.; Greenfield, M. T.; McDermid, S. A.; Dunham, S. U.; Bulte, J. W.; Amedio, J. C., Jr.; Looby, R. J.; Supkowski, R. M.; Horrocks, W. D., Jr.; McMurry, T. J.; Lauffer, R. B. The interaction of MS-325 with human serum albumin and its effect on proton relaxation rates. J. Am. Chem. Soc. 2002, 124, 3152-3162.
(46) Caravan, P. Strategies for increasing the sensitivity of gadolinium based MRI contrast agents. Chem. Soc. Rev. 2006, 35, 512-523.
(47) Aime, S.; Castelli, D. D.; Crich, S. G.; Gianolio, E.; Terreno, E. Pushing the sensitivity envelope of lanthanide-based magnetic resonance imaging (MRI) contrast agents for molecular imaging applications. Acc. Chem. Res. 2009, 42, 822-831.
(48) Tu, C.; Osborne, E. A.; Louie, A. Y. Activatable T1 and T2 magnetic resonance imaging contrast agents. Ann. Biomed. Eng. 2011, 39, 1335-1348.
(49) Major, J. L.; Parigi, G.; Luchinat, C.; Meade, T. J. The synthesis and in vitro testing of a zinc-activated MRI contrast agent. Proc. Natl. Acad. Sci. U.S.A. 2007, 104, 13881-13886.
(50) Zhang, X. A.; Lovejoy, K. S.; Jasanoff, A.; Lippard, S. J. Water-soluble porphyrins as a dual-function molecular imaging platform for MRI and fluorescence zinc sensing. Proc. Natl. Acad. Sci. U.S.A. 2007, 104, 10780-10785.
(51) Louie, A. Y.; Huber, M. M.; Ahrens, E. T.; Rothbacher, U.; Moats, R.; Jacobs, R. E.; Fraser, S. E.; Meade, T. J. In vivo visualization of gene expression using magnetic resonance imaging. Nat. Biotechnol. 2000, 18, 321-325.
(52) Urbanczyk-Pearson, L. M.; Meade, T. J. Preparation of magnetic resonance contrast agents activated by beta-galactosidase. Nat. Protoc. 2008, 3, 341-350.
(53) Frullano, L.; Tejerina, B.; Meade, T. J. Synthesis and characterization of a doxorubicin-Gd(III) contrast agent conjugate: a new approach toward prodrug-procontrast complexes. Inorg. Chem. 2006, 45, 8489-8491.
(54) Lee, J.; Burdette, J. E.; MacRenaris, K. W.; Mustafi, D.; Woodruff, T. K.; Meade, T. J. Rational design, synthesis, and biological evaluation of progesterone-modified MRI contrast agents. Chemistry & Biology 2007, 14, 824-834.
(55) Yang, H.; Zhuang, Y.; Sun, Y.; Dai, A.; Shi, X.; Wu, D.; Li, F.; Hu, H.; Yang, S. Targeted dual-contrast T1- and T2-weighted magnetic resonance imaging of tumors using multifunctional gadolinium-labeled superparamagnetic iron oxide nanoparticles. Biomaterials 2011, 32, 4584-4593.
(56) Perez, J. M.; Josephson, L.; O'Loughlin, T.; Hogemann, D.; Weissleder, R. Magnetic relaxation switches capable of sensing molecular interactions. Nat. Biotechnol. 2002, 20, 816-820.
(57) Kaittanis, C.; Boukhriss, H.; Santra, S.; Naser, S. A.; Perez, J. M. Rapid and sensitive detection of an intracellular pathogen in human peripheral leukocytes with hybridizing magnetic relaxation nanosensors. PloS One 2012, 7, e35326.
(58) Kaittanis, C.; Santra, S.; Perez, J. M. Role of nanoparticle valency in the nondestructive magnetic-relaxation-mediated detection and magnetic isolation of cells in complex media. J. Am. Chem. Soc. 2009, 131, 12780-12791.
(59) Kaittanis, C.; Santra, S.; Santiesteban, O. J.; Henderson, T. J.; Perez, J. M. The assembly state between magnetic nanosensors and their targets orchestrates their magnetic relaxation response. J. Am. Chem. Soc. 2011, 133, 3668-3676.
(60) Haun, J. B.; Castro, C. M.; Wang, R.; Peterson, V. M.; Marinelli, B. S.; Lee, H.; Weissleder, R. Micro-NMR for Rapid Molecular Analysis of Human Tumor Samples. Sci. Trans. Med. 2011, 3, 71ra16.
(61) Asati, A.; Santra, S.; Kaittanis, C.; Nath, S.; Perez, J. M. Oxidase-like activity of polymer-coated cerium oxide nanoparticles. Angew. Chem. Int. Ed. Engl. 2009, 48, 2308-2312.
(62) Asati, A.; Santra, S.; Kaittanis, C.; Perez, J. M. Surface-charge-dependent cell localization and cytotoxicity of cerium oxide nanoparticles. ACS Nano 2010, 4, 5321-5331.
(63) Santra, S.; Kaittanis, C.; Perez, J. M. Aliphatic hyperbranched polyester: a new building block in the construction of multifunctional nanoparticles and nanocomposites. Langmuir 2010, 26, 5364-5373.
(64) Lattuada, L.; Lux, G. Synthesis of Gd-DTPA-cholesterol: a new lipophilic gadolinium complex as a potential MRI contrast agent. Tetrahedron Lett. 2003, 44, 3893-3895.
(65) Weinmann, H. J.; Brasch, R. C.; Press, W. R.; Wesbey, G. E. Characteristics of gadolinium-DTPA complex: a potential NMR contrast agent. Am. J. Roentgenol. 1984, 142, 619-624.
(66) McCarthy, J. R.; Perez, J. M.; Bruckner, C.; Weissleder, R. Polymeric nanoparticle preparation that eradicates tumors. Nano Lett. 2005, 5, 2552-2556.
(67) Sun, E. Y.; Josephson, L.; Weissleder, R. “Clickable” nanoparticles for targeted imaging. Mol. Imaging 2006, 5, 122-128.
(68) Perez, J. M.; Asati, A.; Nath, S.; Kaittanis, C. Synthesis of biocompatible dextran-coated nanoceria with pH-dependent antioxidant properties. Small 2008, 4, 552-556.

It should be borne in mind that all patents, patent applications, patent publications, technical publications, scientific publications, and other references referenced herein and in the accompanying appendices are hereby incorporated by reference in this application to the extent not inconsistent with the teachings herein.

It is important to an understanding to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. An activatable probe comprising:
a superparamagnetic core;
a polymeric matrix coating the metal oxide core; and
a paramagnetic agent encapsulated within the polymeric matrix;
wherein the polymeric matrix is configured to release the paramagnetic agent when subjected to a medium having a pH less than a normal physiological pH.

2. The activatable probe of claim 1, wherein the superparamagnetic core comprises iron oxide.

3. The activatable probe of claim 1, wherein the polymeric matrix comprises a member selected from the group consisting of polyacrylic acid (PAA), dextran, and chitosan.

4. The activatable probe of claim 3, wherein the polymeric matrix comprises polyacrylic acid (PAA).

5. The activatable probe of claim 1, wherein the paramagnetic agent comprises a Gd (gadolinium)-DPTA (diethylenetriaminepentacetate) complex.

6. The activatable probe of claim 1, further comprising a targeting agent having an affinity for a predetermined molecular target encapsulated within the polymeric matrix.

7. The activatable probe of claim 6, wherein the targeting agent is selective for a cancer cell having a pH environment with less than the normal physiological pH.

8. The activatable probe of claim 7, wherein the targeting agent comprises folic acid.

9. The activatable probe of claim 7, further comprising a biologically active agent within encapsulated within the polymeric matrix.

10. The activatable probe of claim 7, wherein the biologically active agent comprises an anti-cancer agent.

11. The activatable probe of claim 10, wherein the anti-cancer agent is selected from the group consisting of taxol and doxorubicin.

12. The activatable probe of claim 11, wherein the anti-cancer agent is conjugated to the paramagnetic agent.

13. The activatable probe of claim 12, wherein the anti-cancer agent is bonded to the paramagnetic agent by a disulfide bond.

14. The activatable probe of claim 1, wherein the normal physiological pH is about 7.4.

15. A method of enhancing imaging sensitivity of tissue in a subject comprising administering to the subject an effective amount of an activatable probe of claim 1 for a time sufficient to release the paramagnetic agent from the polymeric matrix, and subjecting the subject to an magnetic resonance imaging technique.

16. The method of claim 15, wherein the biologically active agent is an anti-cancer agent, wherein the probe further comprises a targeting agent having an affinity for a cancer cell having a pH environment with a less than the normal physiological pH.

17. The method of claim 17, wherein the pH environment of the cancer cell is from about pH 5 to about pH 6.

18. A method of imaging a release of a biologically active agent in a subject comprising administering to the subject an effective amount of an activatable probe of claim 9, and subjecting the subject to an magnetic resonance imaging technique.
